(19) United States
(12) Patent Application Publication
(10) Pub. No.: US 2016/0228652 A1
(43) Pub. Date: Aug. 11, 2016

(54) NEEDLE RETRACTION MECHANISM FOR AUTOINJECTOR

(71) Applicant: MEDIMOP MEDICAL PROJECTS LTD., Ra'anana (IL)

(72) Inventors: Oz CABIRI, Macabim-Reut (IL); Ran HEZKIAHU, Herzliya (IL)

(21) Appl. No.: 15/022,991

(22) PCT Filed: Sep. 30, 2014
(86) PCT No.: PCT/US14/58446

§ 371 Date: Mar. 18, 2016

Related U.S. Application Data
(63) Continuation-in-part of application No. 14/186,403, filed on Feb. 21, 2014.
Application No. 14/186,403 is a continuation-in-part of application No. 14/096,977, filed on Dec. 4, 2013.
(60) Provisional application No. 62/019,066, filed on Jun. 30, 2014.
(60) Provisional application No. 61/884,597, filed on Sep. 30, 2013.

(51) Int. Cl. A61M 5/32; A61M 5/20

(52) U.S. Cl. CPC A61M 5/3234; A61M 5/20; A61M 2005/2013

(57) ABSTRACT

An aspect of some embodiments of the present invention relates to a needle safety mechanism for an autoinjector. Optionally, an unshielded needle may be safeguarded when there is a change in resistance discharging the injector payload. The needle may optionally be safeguarded by retraction to a protected space and/or by deployment of a needle shield. Optionally the stress that prompts safeguarding the needle may include a torque and/or a linear stress.

### BACKGROUND OF THE INVENTION

The present invention, in some embodiments thereof, relates to a needle point safeguarding mechanism and, more particularly, but not exclusively, to a retraction mechanism of an autoinjector.

U.S. Pat. No. 7,530,964 to Cabiri discloses a needle device having a needle retraction mechanism that retracts the needle upon removing the device from the skin surface (either intentionally or unintentionally). Once the needle is retracted, the device is rendered inoperative. The needle can be further made inoperative by bending it when one attempts to reuse the device. In another embodiment, a needle opening formed in the base of the housing can be covered to render the needle inoperative when one attempts to reuse the device. In another embodiment, the needle device instead has a needle shield that automatically covers the needle after use.

U.S. Pat. No. 8,348,898 to Cabiri, discloses a needle assembly adapted for fluid communication with a vial containing a substance to be delivered to a subject, the needle assembly including a needle held in a needle holder, the needle holder confined to move in a housing, and an activation mechanism for activating delivery of the substance through the needle, the activation mechanism including a safety latch that initially impedes movement of the needle holder, wherein when the safety latch is placed on the subject, the safety latch moves to a position that permits moving the needle holder to cause the needle to protrude outwards of the housing to pierce the subject to allow administration of the substance to the subject, characterized by a biasing device arranged to apply a biasing force on the needle to cause the needle to protrude outwards of the housing to pierce the subject, and needle release apparatus including a needle arrestor that initially blocks movement of the biasing device and which releases the biasing device when the safety latch moves to the position that permits moving the needle holder to cause the needle to protrude outwards of the housing.

U.S. Patent Application Publication No. 2009/093,792 to Gross and Cabiri discloses an apparatus for administering a substance to a subject. A vial contains the substance and a stopper is disposed within the vial and is slidably coupled to the vial. A first threaded element is (a) rotatable with respect to the vial and (b) substantially immobile proximally with respect to the vial during rotation of the first threaded element. A second threaded element is threadedly coupled to the first threaded element. At least a distal end of the second threaded element is substantially non-rotatable with respect to the vial, and the distal end of the second threaded element defines a coupling portion that couples the second threaded element to the stopper. The first threaded element, by rotating, linearly advances the stopper and at least the distal end of the second threaded element toward a distal end of the vial. Other embodiments are also described.

Additional background art includes International Patent Application Publication No. WO/2013/104414 to SANOFI-AVENTIS DEUTSCHLAND GMBH, U.S. Patent Application Publication No. 2011/0166509 to Gross and Cabiri, U.S. Patent Application Publication No. 2012/0130344 to Ebbett, U.S. Pat. No. 8,267,890 to Alchas and U.S. Patent Application Publication No. 2009/0012494 to Yeshurun.

### SUMMARY OF THE INVENTION

According to an aspect of some embodiments of the present invention there is provided a drug injector having a needle safeguard mechanism including a housing with a shielded location; a point of the needle protruding from the housing; a retractor mechanically coupled to the needle, the retractor retracting the point to the shielded location; and a sensor operationally linked to the retractor, the sensor sensing a resistance to discharging of the drug through the needle; wherein the retractor is responsive to the sensor to retract the point to the shielded location in response to the resistance.

According to some embodiments of the invention, said retractor includes a support of the needle and the support reverts to a retracted state in response to the resistance.

According to some embodiments of the invention, the support includes an interference element, and the resistance overcomes the interference element.

According to some embodiments of the invention, the interference element includes an annular snap.

According to some embodiments of the invention, the support includes a telescoping assembly.

According to some embodiments of the invention, the telescoping assembly is held in an extended mode by an interference element.

According to some embodiments of the invention, the drug injector further includes a driver driving the discharging and the driver is supported by the support, and in response to the resistance the driver produces a stress on the support and the stress reverts the support to the retracted state.

According to some embodiments of the invention, the drug injector further includes a plunger and the driver drives the discharging by imparting a linear motion to the plunger and the resistance is to the linear motion of the plunger.

According to some embodiments of the invention, the drug injector further includes a lock mechanically coupled to the needle, the lock retaining the point in the protruding position; and wherein the retractor is operationally linked to the lock to release the lock in response to the resistance.

According to some embodiments of the invention, the sensor is further sensitive to a second condition, and the lock retains the point in the protruding position until the second condition is met.

According to some embodiments of the invention, the point is biased to the shielded position.

According to some embodiments of the invention, the lock includes at least one element selected from the group consisting of an interference element, an annular snap, a rib snap, a flange and a groove.

According to some embodiments of the invention, the second condition is a volume of the discharging passing a predetermined threshold.

According to some embodiments of the invention, the sensor senses a torque.

According to some embodiments of the invention, the retracting is in response to a change in the resistance.

According to some embodiments of the invention, the retracting is in response to an increase in the resistance.

According to some embodiments of the invention, the retracting is in response to a decrease in the resistance.

According to some embodiments of the invention, the retracting is in response to the resistance passing a threshold.

According to an aspect of some embodiments of the present invention there is provided a method of safeguarding a needle of an injector comprising: discharging a drug through the needle; sensing a resistance to the discharging, and safeguarding the needle in response to the resistance.

According to some embodiments of the invention, the safeguarding is in response to the resistance passing a predetermined threshold.

According to some embodiments of the invention, the safeguarding is in response to a change in the resistance.

According to some embodiments of the invention, the safeguarding is in response to an increase in the resistance.

According to some embodiments of the invention, the safeguarding is in response to a decrease in the resistance.

According to some embodiments of the invention, the method further includes locking the needle in an extended position during the discharging; and the safeguarding includes releasing the locking.

According to some embodiments of the invention, the resistance occurs upon at least one event selected from the group consisting of a completion of the discharging, a fluid leak, jamming of a mechanical component, and disconnection of a mechanical component and obstruction of a fluid flowpath.

According to some embodiments of the invention, the method further includes inhibiting the safeguarding until a second condition is met.

According to some embodiments of the invention, the second condition includes the discharging passing a predetermined volume.

According to some embodiments of the invention, the volume of discharging is measured by counting motor rotations.

According to some embodiments of the invention, the needle is a component of a pre filled syringe inside autoinjector and the method further includes: placing the autoinjector on a patient; extending the needle to an extended position subsequent to the placing and prior to the discharging, and the safeguarding includes retracting the needle to a retracted position.

According to some embodiments of the invention, the method further includes indicating that it is safe to remove the autoinjector after the retracting.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart illustrating a method of safeguarding a needle of an autoinjector according to an embodiment of the present invention;

FIG. 2 is a illustrating a method of injecting a drug according to an embodiment of the present invention;

FIG. 3 is a state diagram of an autoinjector according to an embodiment of the present invention;

FIGS. 4A-D are schematic illustrations of a needle safeguarding mechanism that is triggered by a linear force resulting from resistance to discharge of a drug according to an exemplary embodiment of the present invention;

FIGS. 5A-C are schematic side view illustrations of an autoinjector with a needle safeguarding mechanism including concentric cylindrical telescoping elements according to an exemplary embodiment of the present invention;

FIGS. 6A-C are schematic side view illustrations of a lock of a needle safeguarding mechanism triggered by a torque according to an embodiment of the present invention;

FIGS. 7A,B illustrate a schematic side view and a top cross-sectional view respectively of a needle safeguarding mechanism triggered by a torque according to an embodiment of the present invention;

FIGS. 8A,B illustrate a schematic side view and a top cross-sectional view respectively of a needle safeguarding mechanism triggered by a torque and a second condition according to an embodiment of the present invention;

FIG. 9A-F are detailed schematic views illustrating states of an autoinjector according to an embodiment of the present invention;

FIGS. 10A-E illustrate a needle safeguarding mechanism triggered by a torque and a linear force according to an embodiment of the present invention;

FIGS. 11A-C illustrate a manual trigger for a needle protector of an autoinjector according to an embodiment of the present invention;

FIGS. 12A,B illustrate an optional non-circular cross section for syringe according to an embodiment of the present invention; and

FIGS. 13A-D illustrate an autoinjector with a motor switch located in the main body of the injector in accordance with some embodiments of the current invention.

### DESCRIPTION OF SPECIFIC EMBODIMENTS OF THE INVENTION

The present invention, in some embodiments thereof, relates to a needle point safeguarding mechanism and, more particularly, but not exclusively, to a retraction mechanism of an autoinjector.

### OVERVIEW

### 1 Needle Protection Mechanism

An aspect of some embodiments of the present invention relates to a needle safety mechanism for an autoinjector. Optionally, an unshielded needle may be safeguarded in response to resistance and/or a change of resistance to discharging of the injector payload.

In an exemplary embodiment, a lock may hold a needle in an unshielded position. The lock may optionally act as a support for a discharge driver. Increasing resistance to discharge may increase the stress on the driver. When the stress on the driver increases beyond a threshed and/or decreases beyond a threshold the lock may be released and/or the needle retracted to a safe location. For the sake of the current disclosure retraction of a needle may include pulling a needle point back into a shielded location without changing the length and/or shape of the shielding; and/or retracting may include extending the housing of the injector and/or a shield ahead of a needle point such that the needle is left in a shielded location.

In some embodiments, a support for a needle assembly may include a telescoping assembly. Optionally, the telescoping assembly may retract the needle (for example by contracting) in response to a stress from a driver.

In some embodiments, the safeguarding mechanism may include a sensor sensitive to a linear force from the driver. For example, a pushing force passes a threshold, a lock may be released moving the needle to the retracted configuration.

In some embodiments, the force to insert the needle to the skin of a patient may range for example between 0.1 to 0.5 N. Optionally, the force required to inject the drug (for example the force on a syringe plunger) may range for example between 5 to 20 N.

In some embodiments a needle protection mechanism may be triggered by a linear force greater than, for example, between 10 to 30 N.

For example, an autoinjector may be activated by manually pushing with enough force to insert the needle. The device may then apply an injection force to inject a drug. Once the entire drug is injected and/or when there is an obstruction and/or occlusion, the injection force may rise until it passes a threshold triggering safeguarding of the needle and/or ending injection.

For example in the event of an occlusion and/or at the end of delivery, the linear force generated by the device may increase to the level of up to 60 N. A needle safeguarding mechanism may include a sensor that is sensitive to the force. For example the sensor may include a snap that gives way at 40 N returning the needle to the retracted position.

In some embodiments, the stress to inject a medicine and/or to trigger safeguarding of a needle may include a torque. For example, injection of medicine may be driven by a plunger. The plunger may optionally be driven by a threaded assembly, for example a threaded screw and/or teeth and/or a telescoping assembly. Optionally the pitch of the teeth and/or an associated screw may range for example between 0.5 and 2 mm. The diameter of the screw may range for example between 3 and 12 mm. The torque to power injection may range for example between 0.2 and 1.0 N*cm. The trigger torque (the torque at which the needle safeguarding is triggered) may range for example between to 2-4 N*cm.

In some embodiments a safety mechanism may include linear movement of the ranging between 5 to 15 mm. For example movement of the safety mechanism may include extension of a needle during insertion and/or retraction of the needle and/or extensions of a safety shield and/or retraction of a safety shield. Optionally a needle insertion length (for example the length of needle inserted into a patient) may range for example between 5 to 10 mm.

During injection, the linear movement of a plunger may range for example between 10-50 mm. The length of movement of the plunger may vary for example with the volume of medicine to be injected that may range for example between 0.5 to 3 ml.

In some embodiments, a sensor of a safeguarding mechanism may be sensitive to a torque. For example, the needle may be retracted when the mechanism is exposed to a twisting moment. Optionally, discharge may be driven by a torque. For example the driver may apply torque to threaded element pushing a plunger. When the torque on the driver reaches a threshold value, the needle may be released and/or retracted and/or a needle shield may be deployed.

In some embodiments an interference element (for example a snap) may provide resistance to retraction. For example, an annular ring may impede contraction of a telescoping assembly. Alternatively or additionally a rib may impede twisting of a support structure. When stress from the driver passes a threshold, the stress may optionally overcome the interference element. Overcoming the interference element may for example revert the support to a retracted configuration.

In some embodiments, a stress resulting from resistance to discharge may trigger deployment of a needle shield. The needle shield may optionally move to shield the needle in reaction to the increased stress.

### 2 Manual Needle Safety Retraction Mechanism

An aspect of some embodiments of the present invention relates to a manual retraction mechanism for an autoinjector. The manual retraction mechanism may allow a user to retract the needle of the autoinjector to a safe position in case of for example a malfunction of the injector. For example the safety retraction mechanism may include a release switch. When activated by the user, the release switch may permanently retract the needle into the injector. Alternatively or additionally the release switch may trigger deployment of a needle shield. For example, the casing of the injector may be biased to an extended configuration covering the needle. When the autoinjector is activated the casing may locked in a contracted configuration unshielding the needle. When the user pushes a switch, the lock may be permanently released, covering the needle and preventing reuse and/or a needle stick hazard.

### 3 Flexible Cover to Peel Adhesive Protector

An aspect of some embodiments of the present invention relates to a mechanism to peel an adhesive protector when a safety cover is pulled off of an autoinjector. For example the safety cover may be flexible and/or a hinged and/or may be anchored to an edge of the adhesive cover. As the safety cover is pulled away the pulling force may be transferred to a peeling force at the edge of the adhesive protector.

### 4 Stabilized Manually Held Autoinjector

An aspect of some embodiments of the present invention relates to a manually held autoinjector with stabilization. For example, while in use the auto injector may be held to the skin of a patient by a user, for example the user may be a caretaker and/or the patient himself. In some cases, it may be difficult for a user to manually hold an autoinjector immobile enough and/or for long enough to complete injection. The auto injector may include an adhesive to increase stability of the injector. The adhesive may hold the activation zone of the injector and/or the injection zone and/or a needle aperture and/or a needle stable with respect to the skin of the patient.

In some embodiments, a syringe may be held substantially perpendicular to a surface contacting the user's skin. For example, the syringe may be held at an angle ranging between 75° to 105° to the user's skin during injection. For example, holding the injector immobile may include holding a needle at an injection depth of between 5 to 10 mm. For example, holding the injector immobile may include not sliding the injector along the patient's skin and/or straining the needle enough to bend and/or occlude the needle.

In some embodiments the injector may be held substantially immobile with respect to the skin of a patient for a time ranging for example between 10 to 600 seconds and/or the injector may be held substantially immobile with respect to the skin of a patient for a time ranging for example between 30 to 180 seconds. For example the injector may inject of volume of drug ranging between 0.5 to 3 ml.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Embodiments

### 1 Method of Safeguarding a Needle

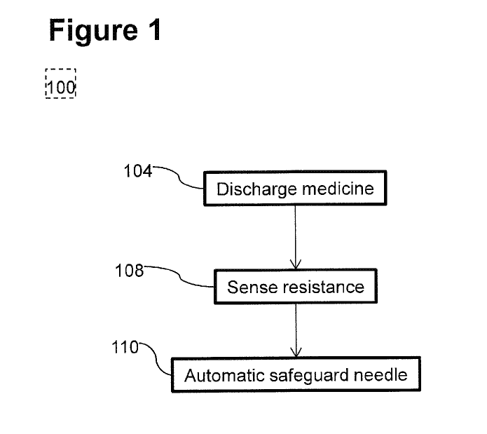

Referring now to the drawings, FIG. 1 illustrates a method of safeguarding a needle of an autoinjector according to an embodiment 100 of the present invention. In the exemplary method, when medicine is being discharged 104 from the autoinjector there may be a resistance to the discharging 104. The resistance may be sensed 108. When the resistance reaches a threshold level the needle may be safeguarded 110.

In some embodiments of an autoinjector, the resistance to discharge may change at some point during the injection. For example, there may be an increase in resistance to movement of a plunger when it reaches the end of a syringe. For example, there may be an increase in resistance to movement of a plunger when a fluid path becomes obstructed. The change in resistance may be sensed 108 by, for example, a pressure sensitive trigger. Optionally increased pressure from the plunger may, trigger safeguarding 110 the needle. For example safeguarding 110 may include retracting the needle tip to a shielded location and/or movement of a shield to cover the needle tip.

### 2 Method of Injecting a Drug

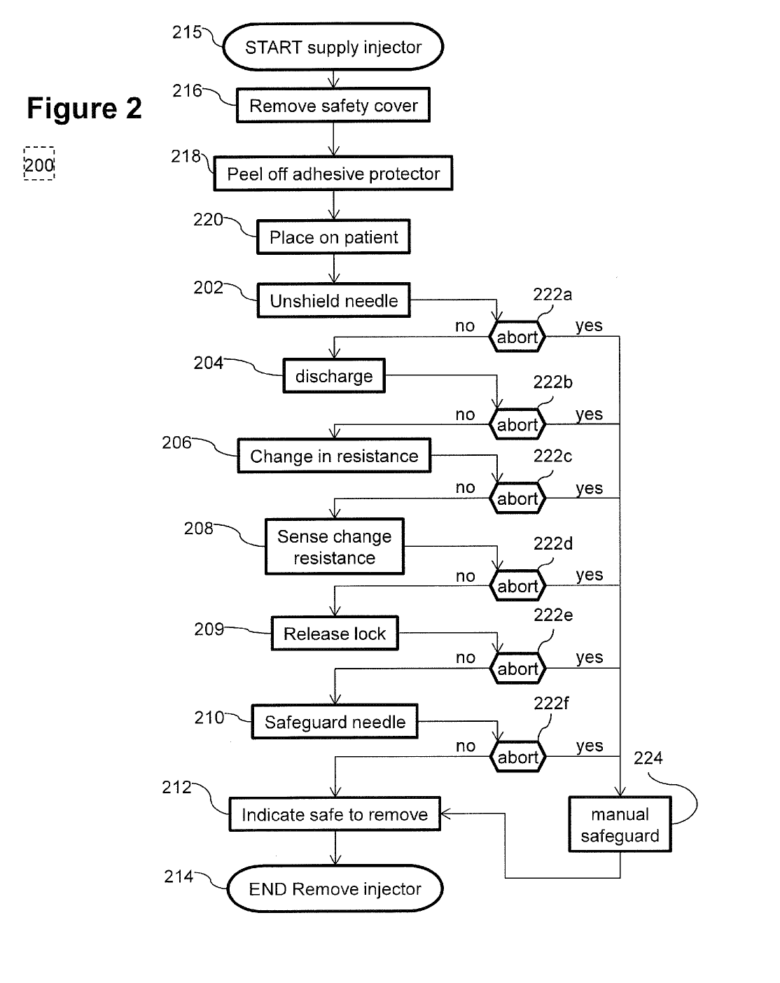

Referring now to the drawings, FIG. 2 illustrates a method of injecting a drug according to an embodiment 200 of the present invention. In the exemplary method, a needle is optionally retracted to a safeguarded position in response to a resistance to drug discharge. For example, the resistance may increase due to a malfunction such as an obstruction of a flow path and/or the injector completing injection of a full dose.

In exemplary embodiment 200 a user (for example a patient and/or a medical aid in home care) may be supplied 215 with an autoinjector ready to administer a medicine. The user may optionally remove 216 a safety cover from the injector. Removing 216 the cover may also automatically peel 218 an adhesive protector from an adhesive and/or remove a sterile cover from a needle. For example, the adhesive may be supplied to stabilize the injector on the skin of a patient during injection. The injector may optionally be placed 220 on a patient (who may be the user). In some embodiments a user may hold the injector to the skin of a patient, the adhesive may stabilize the injector for example from shifts and/or movements of the patient and/or the user.

In some embodiments, before injection, a needle may optionally be unshielded 202. For example, while placing 220 the injector on the patient, the user may put pressure on an activation mechanism. The activation mechanism may react to pressure for example by unshielding 202 the needle, for example by extending the needle outward and/or inserting the needle into the patient. The needle may optionally be locked in the extended position. Optionally, the needle may be biased to a protected position (for example to retract into a housing of the injector). Alternatively or additionally, the needle may be biased to the unshielded position. Alternatively or additionally, the autoinjector may be supplied with the needle in an extended mode and/or protected by a cover.

At any point during the injection process, a manual safeguard 224 mechanism may be used to place the injector in a safeguarded mode. For example, if the user decides to abort 222a-f at any point in the process (for example if he detects some sort of malfunction and/or feels a negative reaction to the medicine) the user may manually safeguard 224 the needle. For example, a needle shield may be released to manually safeguard 224 the needle and/or the needle may be retracted. Optionally there may be an indicator to indicate 212 whether the needle was automatically safeguarded 210 and/or whether needle was manually safeguarded 224. Alternatively or additionally there may be an indicator whether a full dose was administered and/or how much medicine was administered.

Once the needle is inserted into the patient, the injector may optionally begin discharging 204 medicine. For example the medicine may be injected through the needle into the patient. Optionally, discharge may continue until a full dose of the medicine is administered.

In some embodiments, after administration of a full dose of the medicine, there may be a change 206 in resistance to further discharging. For example in a syringe based injector, a plunger may reach the end of the syringe and cease to move increasing resistance. Alternatively or additionally, after discharging the entire dose a transmission may be disconnected (for example a threaded element may pass the end of its threading) reducing resistance. Alternatively or additionally, the change 206 in resistance may result from another cause for example increased resistance due to a full or partial occlusion of a fluid pathway and/or jamming of a mechanical component (for example cross threading of a screw). The change of resistance may optionally be sensed 208 triggering safeguarding 210 of the needle.

In some embodiments, the needle may be locked in an unshielded 202 state by a force sensitive lock. When the lock senses 208 the change 206 in resistance, it may release 209 the needle which may be safeguarded 210 by moving the tip to a shielded position (for example by retracting the needle tip to a shielded location). Alternatively or additionally, safeguarding 210 may include covering the needle tip with a needle shield.

In some embodiments, a flag may be supplied (for example a LED and/or a changing color indicator) to indicate 212 to the user that the needle has been safeguarded 210 and/or that the injector can safely be removed 214 from the patient.

### 3 States of an Autoinjector

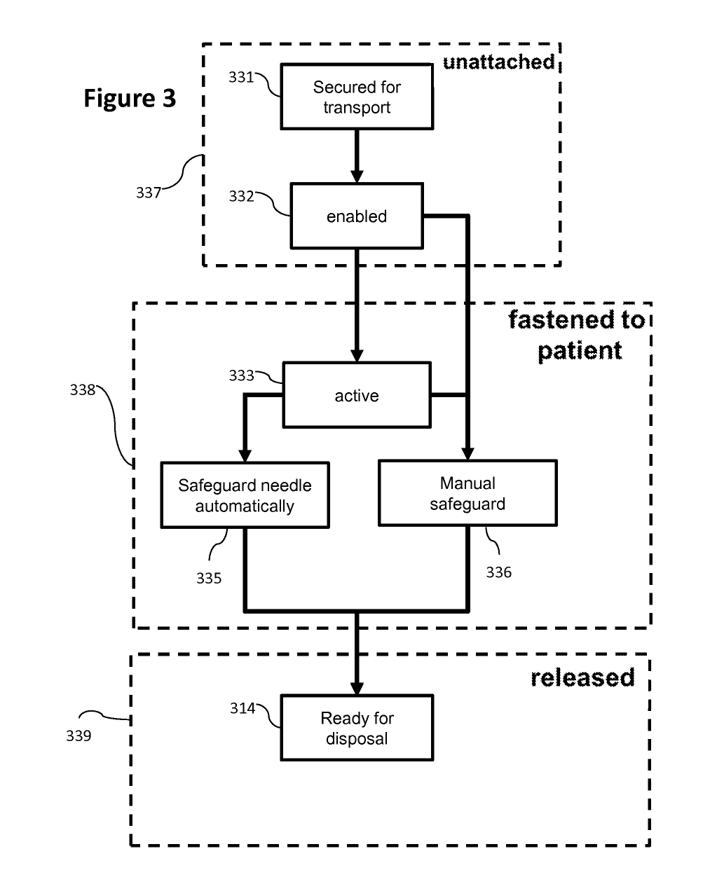

FIG. 3 is a state diagram of an autoinjector according to an embodiment of the present invention. In general, may be supplied in an unattached 337 state. An unattached 337 autoinjector may have a secured 331 state. For example in the secured 331 state the injector may be safe to handle and/or transport. Optionally the injector may have an enabled 332 state. For example, in the enabled 332 state, the injector may be unstable and/or easily activated. For example, an injector may be switched from the secured 331 state to the enabled 332 state by removing a needle protector and/or an adhesive cover.

Once activated the injector may optionally be fastened to a patient. In the fastened 338 state the injected may optionally be activated. For example, while the injector is in the active 333 state, a needle may project from the injector. In some embodiments the injector may be hazardous to handle in the enabled 332 and/or active 333 state.

In some embodiments, after use (optionally whether or not administration of the full dose was successful) the user may want to remove and/or dispose of the autoinjector. In some embodiments, it may be difficult and/or dangerous to remove an injector in the enabled and/or active state. For example, when an injector is fastened to a patient by an adhesive, it may be difficult to remove the needle by pulling the injector away from the skin. Optionally, first a needle may be retracted from the skin into the injector. Subsequently the adhesive may be removal by peeling from the skin. In some embodiments, the injector may automatically be safeguarded 335 for example by retraction of a needle upon completion of injection. Alternatively or additionally, the user may have the option to manually secure the injector into a safeguarded 336 state. For example, the optionally of manually needle retraction may avoid the situation where a patient may not be able to properly remove the injector due to a malfunction that leaves the injector fastened to the skin with the needle inserted into the patient. During and/or after safeguarding 335, 336 the injector may be removed from the patient.

Optionally, the injector may have a final released state 339, for example wherein the needle is retracted back into the injector and/or the needle tip is shielded and/or the injector has been unfastened from the patient. Optionally one or more indicators may be supplied to indicate the state of the injector and/or the quantity of medicine discharged. Once released, the injector may be in final 314 state (protected from hazards and/or ready for disposal, for example in a municipal waste).

### 4 Needle Safeguarding Triggered by a Linear Force

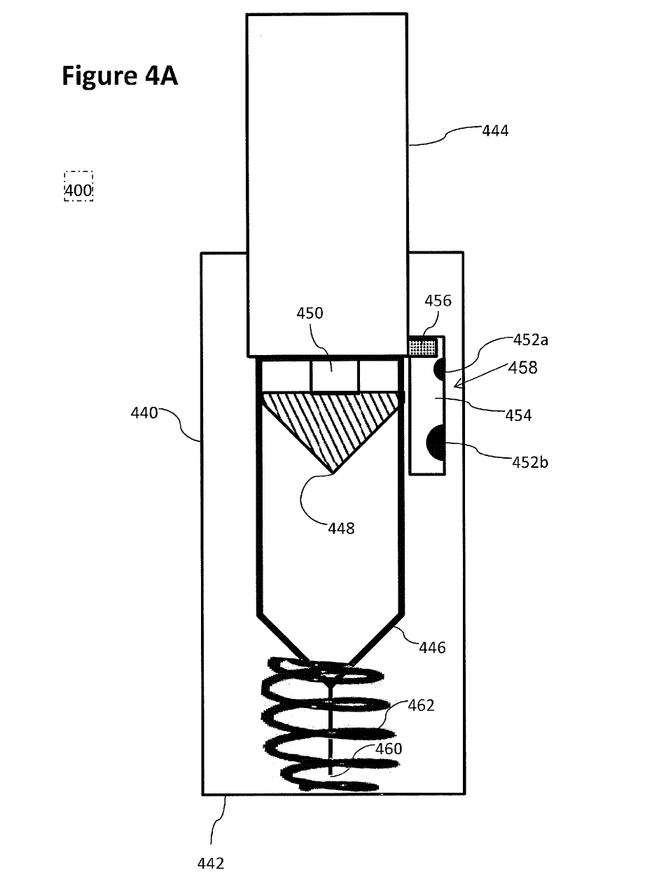

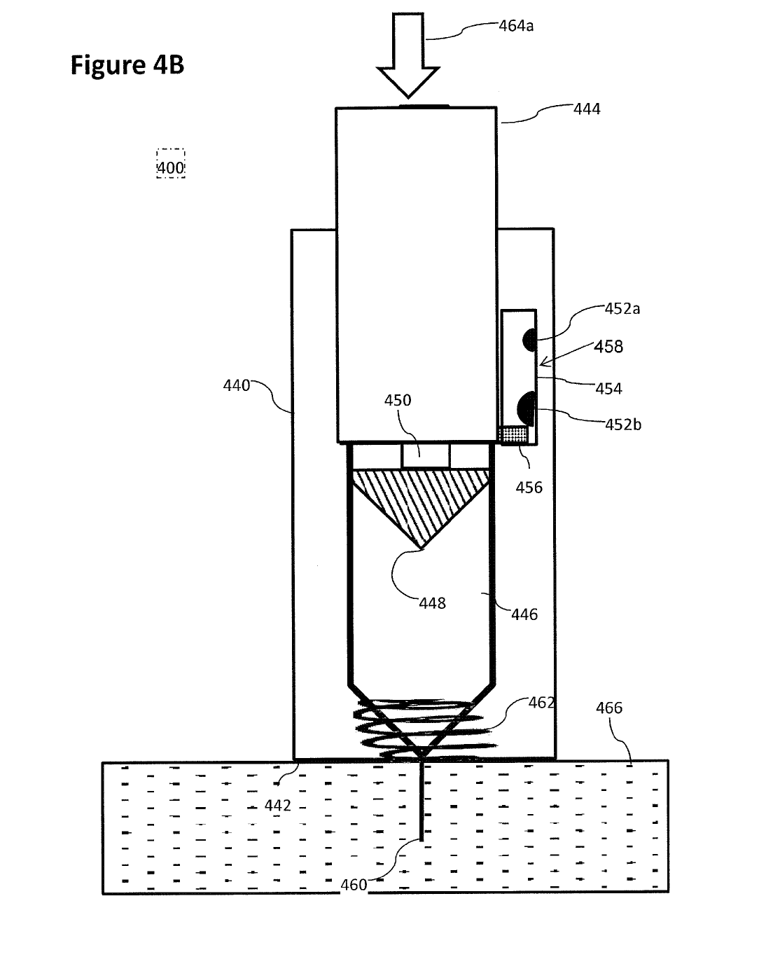

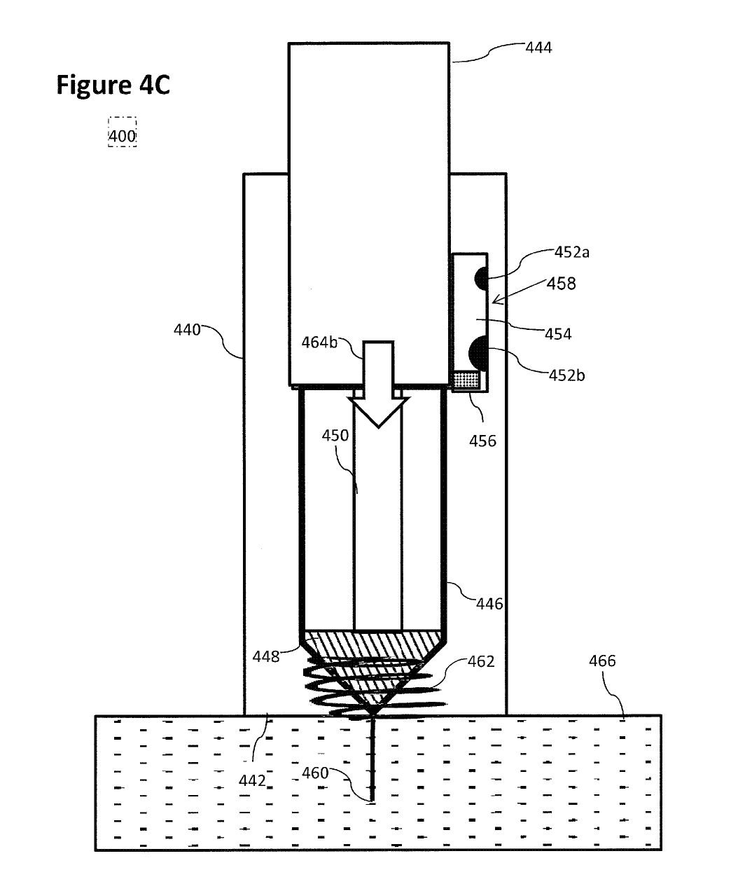

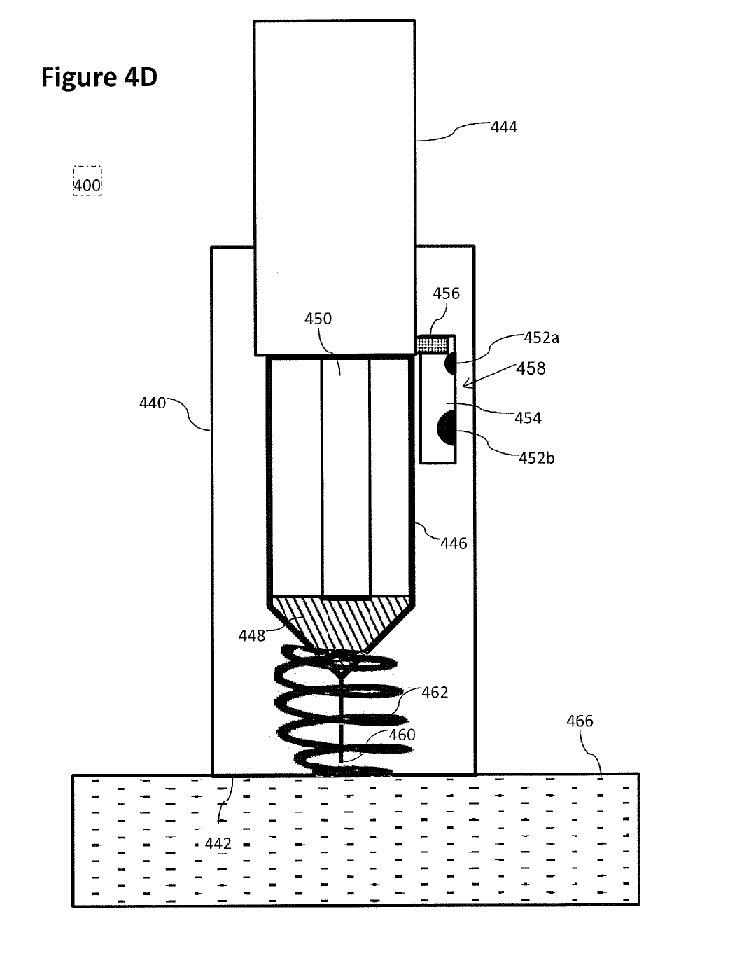

FIGS. 4A-D are schematic illustrations of a needle safeguarding mechanism that is triggered by a linear resistance to discharge of a drug according to an exemplary embodiment 400 of the present invention. In exemplary embodiment 400, a needle is biased to a retracted state for example by a spring 462. During injection (for example as illustrated in FIG. 4B) the needle is optionally held in an unshielded state by a driver 444. Driver 444 itself may be locked in place for example by an interference element 452b. Additionally or alternatively driver 444 may push a plunger 448 to discharge a medicine. When a full dose has been administered (for example as illustrated in FIG. 4C), plunger 448 may optionally reach the end of its path and be stopped. For example plunger 448 may be stopped by the front end of a syringe 446. Optionally syringe 446 may rest against a housing 440. In some embodiments interference element 452b may serve as a sensor. For example, the force of the driver pushing a plunger rod 450, plunger 448 and/or syringe 446 against housing 440 may optionally overcome interference element 452b, unlocking driver 444 and/or syringe 446 and/or the needle (for example as illustrated by force 464b of FIG. 4C). Once unlocked, driver 444 may revert to a retracted state and/or the needle may optionally retract back into the housing (for example as illustrated in FIG. 4D).

In some embodiments, interference elements 452a,b and spring 462 may be elastic and/or deformable, allowing them to be deformed by a sufficient force (for example as illustrated by force 464b of FIG. 4C) to unshield the needle.

FIG. 4A illustrates exemplary embodiment 400 in a deployed state prior to activation. For example, in the deployed state a needle tip 460 is shielded by an activation zone 442 of housing 440. Needle tip 460 is safeguarded by a retraction assembly including for example a spring 462 biasing the needle into housing 440 and a lock 458 and a driver 444 which hold needle tip 460 and its supporting syringe 446 inside housing 440. In exemplary embodiment 400, lock 458 optionally includes a driver support 456 and a slot 454 and interference elements 452a and 452b. In FIG. 4A, driver 444, syringe 446 and needle tip 460 are held in the retracted position by interference element 452a and/or spring 462.

FIG. 4B illustrates exemplary embodiment 400 in an activated state, for example right before and/or during discharge of a medication. For example, embodiment 400 is activated by placing activation zone 442 against the skin 466 of a patient and/or pushing driver 444 with a sufficient force 464a. Pushing driver 444 optionally pushes needle tip 460, syringe 446, and/or driver support 456 from the retracted position (illustrated in FIG. 4A) to the extended position (illustrated in FIG. 4B). As support 456 moves from the retracted to the extended position, it slides along slot 454. Sliding along slot 454, support 456 overcomes biasing spring 462 and/or interference elements 452a,b. Optionally support 456, syringe 446 and/or driver 444 are locked into the unshielded position by interference element 452b. Once activated, driver 444 may apply a force 464b (see FIG. 4C) on plunger rod 450 and/or plunger 448 to discharge medicine.

FIG. 4C illustrates exemplary embodiment 400 at the end of discharge of a medicament. Plunger 448 has optionally reached the end of its path and is encounters resistance from further movement. For example, further movement may be resisted by the front end of syringe 446 which is resting against housing 440. Optionally the resistance force 464b may be sensed by driver 444 as a strong counter force pushing driver 444 and driver support 456 distally away from plunger 448. The counterforce may optionally become large enough to overcome interference element 452b. Overcoming interference element 452b may optionally trigger safeguarding needle tip 460. For example, biasing spring 462 and/or momentum may optionally overcome interference element 452a returning support 456, driver 444, syringe 446 and/or needle tip 460 to the retracted safeguarded position (as illustrated for example in FIG. 4D).

### 5 Needle Safeguarding by a Telescoping Element

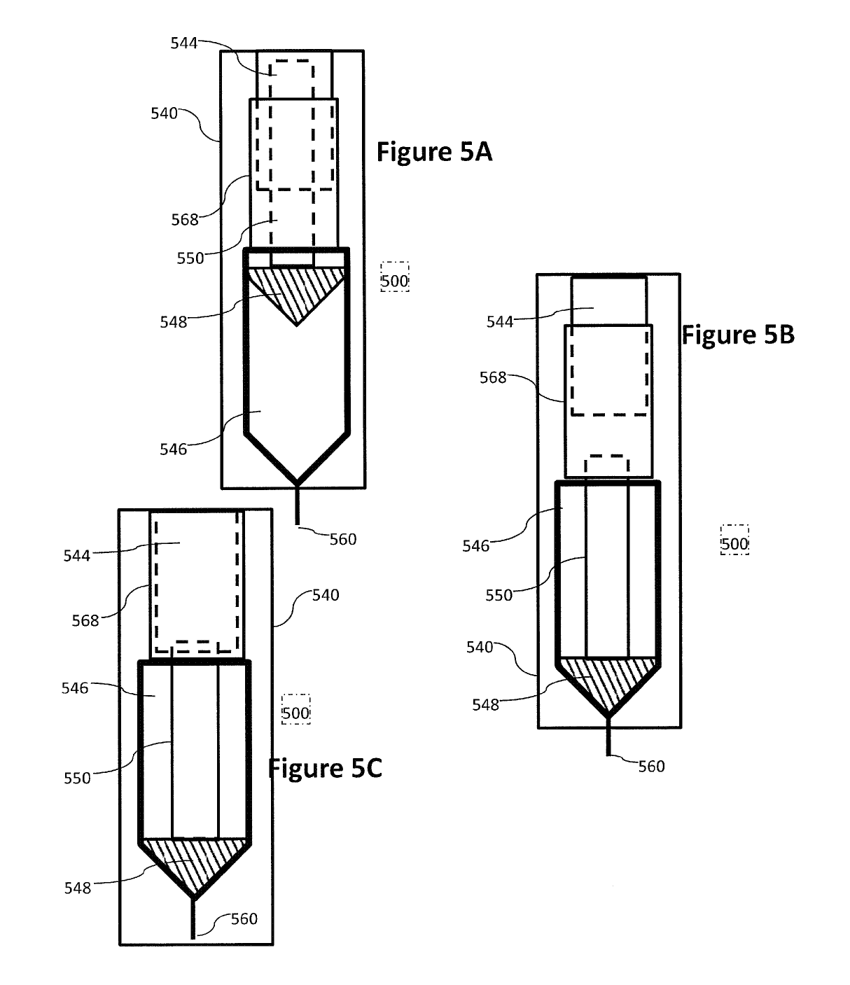

FIGS. 5A-C are schematic side view illustrations of an autoinjector with a needle safeguarding mechanism including concentric cylindrical telescoping elements according to an exemplary embodiment 500 of the present invention. The safeguard mechanism of exemplary embodiment 500 includes a cylindrical driver 544 mounted concentrically in a cylindrical sleeve 568. When driver 544 telescopes out from sleeve 568 a needle tip 560 is unshielded. When driver 544 retracts into sleeve 568, needle tip 560 is safeguarded. As used herein the word cylindrical may include a cylinder with a circular and/or a non-circular cross section.

FIG. 5A illustrates the injector of exemplary embodiment 500 in an activated state before discharge of a medicine payload. Optionally, in the activated state, driver 544 is telescoped out of sleeve 568. Driver 544 pushes, for example, against a housing 540. Sleeve 568 optionally pushes a syringe 546 and/or needle point 560 extending needle point 560 out of housing 540 and/or unshielding needle point 560.

FIG. 51 illustrates exemplary embodiment 500 at the end of medicine discharge. Driver 544 has optionally pushed a plunger rod 550 and/or plunger 548 into syringe 546 discharging the payload of the injector. In the example, further pushing by driver 544 will, for example, create a high stress between driver 544 and plunger rod 550. The stress may optionally cause driver 544 to retract into sleeve 568.

FIG. 5C illustrates that in exemplary embodiment 500, when driver 544 retracted into sleeve 568 needle point 560 is optionally retracted back into a protected position. In the protected position, needle tip 560 may optionally be shielded by housing 540 safeguarding needle tip 560.

### 6 Needle Safeguarding Triggered by a Torque

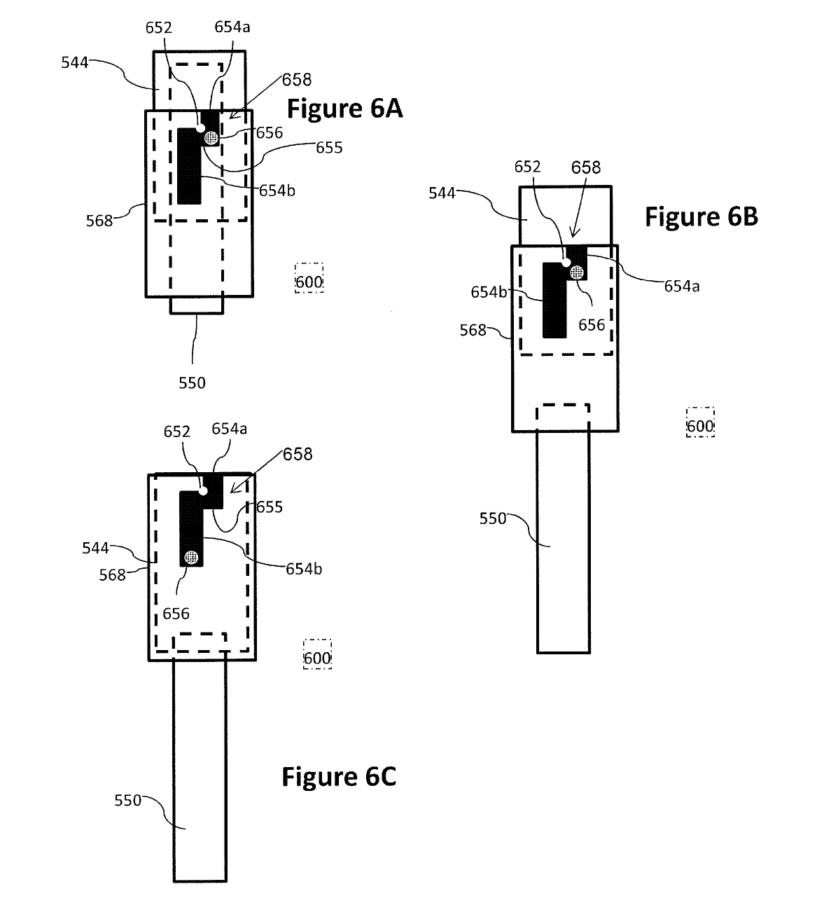

FIGS. 6A-C are schematic side view illustrations of a lock 658 of a needle safeguarding mechanism triggered by a torque according to an embodiment 600 of the present invention. For example, in embodiment 600 lock 658 holds a needle (not shown) in an unshielded and/or extended state. Optionally a torque overcoming an interference element releases lock 658. For example in embodiment 600, when lock 658 is released, a driver 544 slides into a sleeve 568 retracting the needle to a safeguarded location.

The needle safeguarding mechanism of embodiment 600 may optionally include concentric cylindrical telescoping elements similar for example to embodiment 500. Embodiment 600 is illustrated in FIGS. 6A-C in states respectively similar to the states illustrated for embodiment 500 in FIGS. 5A-C. Embodiment 600 may for example be used to hold a needle in an unshielded and/or safeguarded state in a manner similar to embodiment 500.

FIG. 6A illustrates the safeguarding mechanism of exemplary embodiment 600 in an exemplary activated state before discharge of a medicine payload. Optionally, in the activated state, driver 544 is telescoped out of sleeve 568. Driver 544 may optionally be connected to a support 656. Support 656 may for example move along a doglegged slot having two tracks, for example track 654a and track 654b. In the extended state of FIG. 6A support 656 is blocked by a shoulder 655 of track 654a optionally preventing driver 544 from sliding into sleeve 568. Optionally, support 656 is blocked from moving to track 654b by an interference element 652. Optionally interference element 652 may be made of an elastic material that may be overcome by a sufficient stress.

FIG. 6B illustrates exemplary embodiment 600 at the end of medicine discharge. Driver 544 has optionally pushed a plunger rod 550 to the end of a syringe. Pushing of rod 550 may be for example by a telescoping screw. In the example of embodiment 600, further pushing by driver 544 may, for example, create a high torque stress between driver 544 and plunger rod 550 and/or between driver 544 and sleeve 568. The torque may for example overcome interference element 652 and slide support 656 onto track 654b.

FIG. 6C illustrates that in exemplary embodiment 600, after support 656 has been forced by torque onto track 654b. Support 656 may slide along track 654b and/or driver 544 may slide into sleeve 568. Sliding driver 544 into sleeve 568 may optionally revert driver 544 and/or sleeve 568 and/or the needle to a retracted state and/or retract needle point 560 back into a protected position for example as illustrated in FIG. 5C.

In some embodiments there may be biasing element, for example a spring, biasing the needle to a safeguarded state. Optionally, embodiments 500 and/or 600 may contain a biasing element, for example similar to spring 462.

### 7 An Extended Area Rotational Interference Element

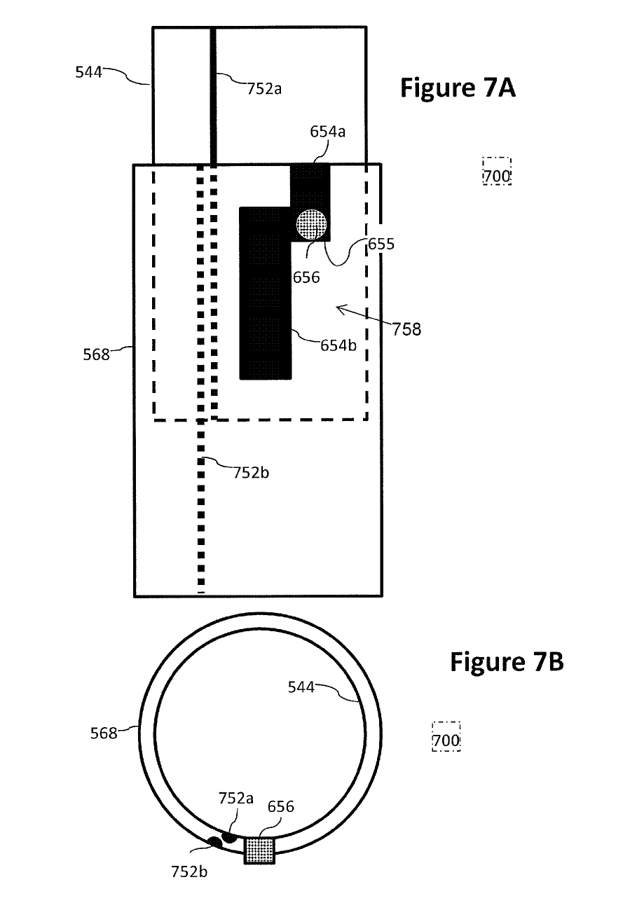

FIGS. 7A,B illustrate a schematic side view and a top cross-sectional view respectively of a needle safeguarding mechanism triggered by a torque according to an embodiment 700 of the present invention. Embodiment 700 includes elongated interference elements 752a,b resisting rotation of driver 544 with respect to sleeve 568. The elongated interference elements 752a,b spread the resistance to rotation over a large area. The large interference elements 752a,b may in some embodiments produce a consistent resistance even if there are small flaws during formation of part elements 752a,b.

Similar to embodiment 600, embodiment 700 includes two concentric cylindrical telescoping elements, a driver 544 in a sleeve 568. A lock 758 includes a driver support 656 that moves between two tracks 654a,b. Lock 758 may optionally move from a locked state (holding the system in an extended mode) to a released state in response to a torque twisting driver 544 with respect to sleeve 568. For example twisting may trigger retraction of a needle. For example twisting may be triggered by a plunger reaching the end of a syringe after administering a full dose of a drug. Alternatively or additionally retraction may be triggered by an obstruction in a fluid pathway providing resistance to movement. The resistance to movement of a screw thread plunger may optionally produce the torque to twist and/or unlock the retractor.

One difference between embodiment 600 and embodiment 700, is the location and geometry of the resistance interference elements. In 700 the resistance interference elements 752a,b include two ribs, one rib (element 752a) located on an outer surface of driver 544 and another rib (element 752b) located on an inner surface of sleeve 568. Resistance interference elements 752a,b resist rotation of a driver 544 within sleeve 568 and/or movement of support 656 from track 654a to 654b. When support 656 is on track 654a, driver 544 and/or the needle may remain in an extended position. When support 656 is on track 654b, driver 544 and/or the needle may be retracted. Alternatively or additionally there may be more than two ribs serving as resistance elements. For example, multiple pairs of ribs may engage simultaneously to create a compound resistance at a single point of rotation. Alternatively or additionally, there may be more than two sections of tracks and or there may be multiple steps of resistance and/or extension and/or retraction.

### 8 Multi-Conditional Needle Safeguarding

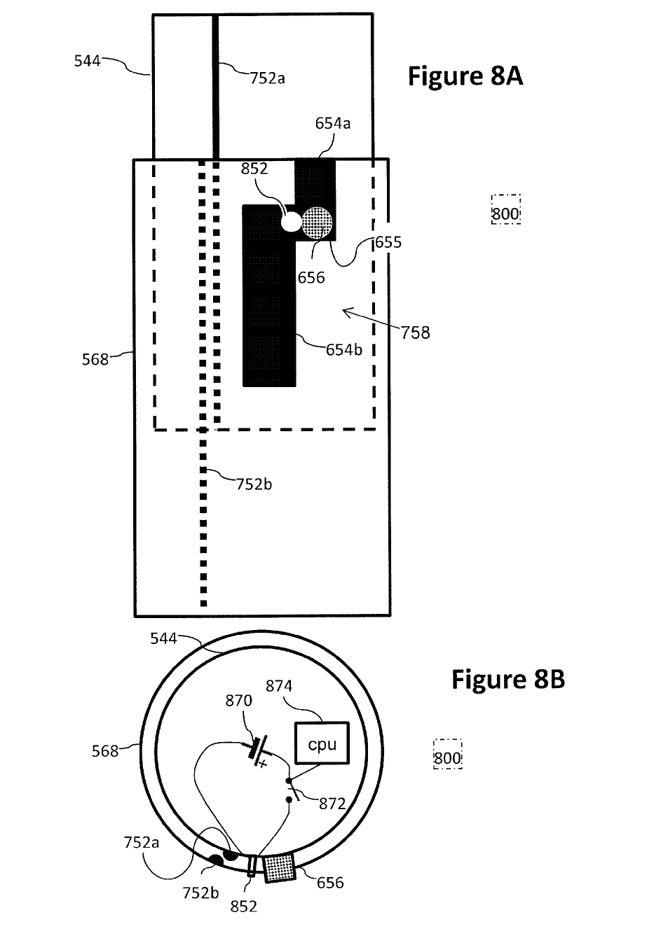

FIGS. 8A,B illustrate a schematic side view and a top cross-sectional view respectively of a needle safeguarding mechanism triggered by a torque and a second condition according to an embodiment 800 of the present invention. Embodiment 800 includes a logical sensor. For example a logic controlled interference element 852 resists rotation of driver 544 with respect to sleeve 568. Interference element 852 may optionally be responsive to a controller 874. In some embodiments even when there is a torque capable of overcoming interference elements 752a,b the needle will not retract until controller 874 opens interference element 852. For example, controller 874 may open resistance element 852 when a predetermined portion (for example substantially all of) a payload of the injector has be administered. Optionally, the needle will be retracted when there is a resistance to discharge of the medicine and the entire payload has been administered. Optionally the needle may not retract in response to an obstruction to the fluid pathway when it occurs before the entire payload is administered.

Embodiment 800 may be similar in many aspects to embodiment 700. Embodiment 800 differs from embodiment 700, at least in that embodiment 800 includes an additional interference element 852. Optionally, element 852 blocks retraction of a needle until a further condition is fulfilled (for example in embodiment 700 the needle may retract on any application of a sufficient torque to driver 544 whereas in embodiment 800 the needle may optionally be prevented from retracted until a sufficient torque is applied and the payload of the injector has been sufficiently administered).

In some embodiments logic controlled resistance element 852 may block movement of support 656 to track 654b. When the sufficient portion of the payload of the injector has been administered, controller 874 may close a switch 872. Closing switch 872 may optionally allow a current from a battery 870 to flow to element 852, melting element 852 and/or allowing support 656 to move to path 654b and/or allowing driver 544 to slide into sleeve 568.

### 9 Detailed Illustration of States of an Injector

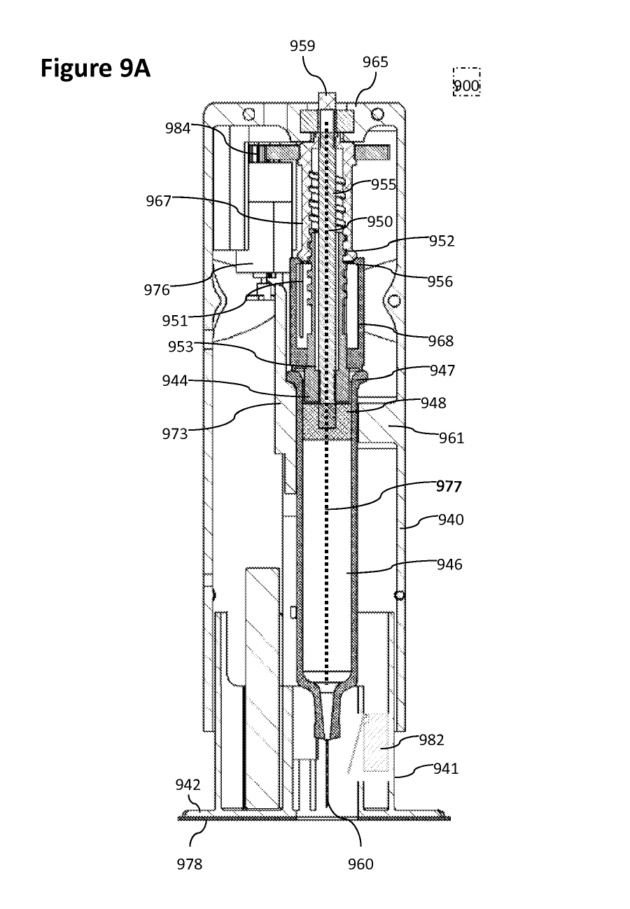

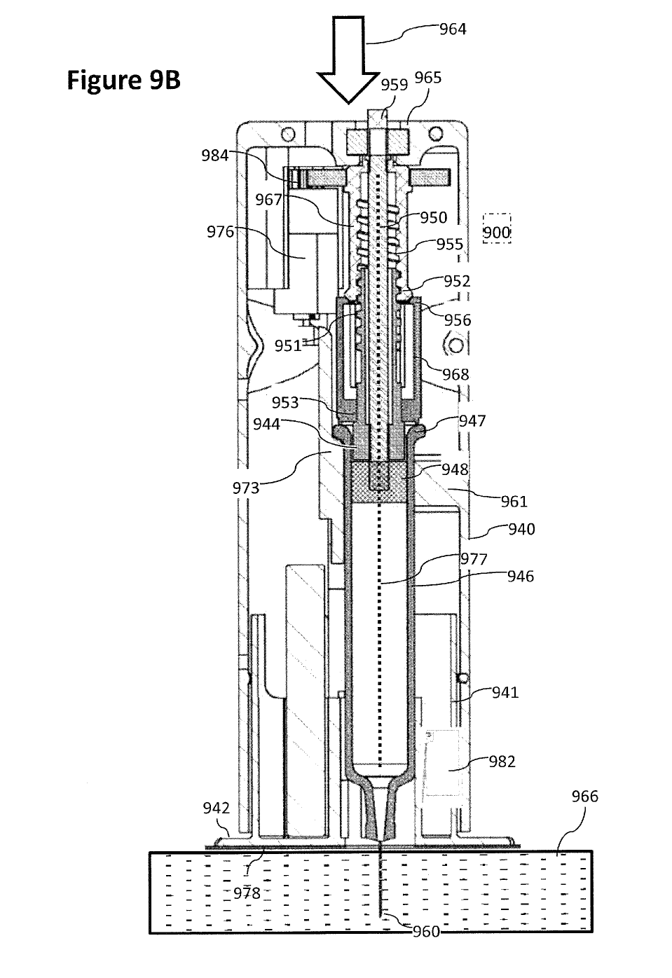

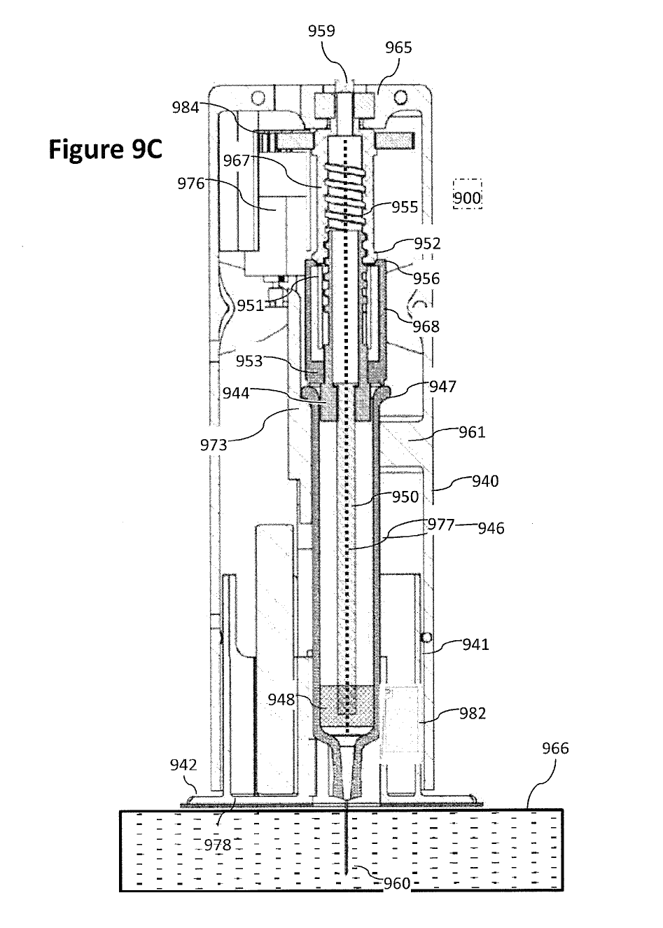

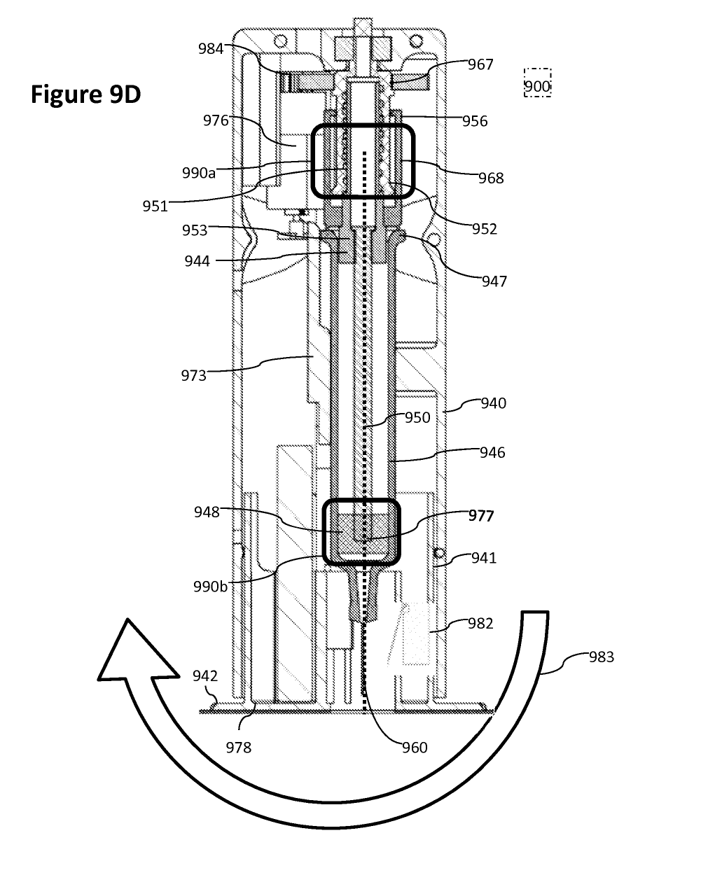

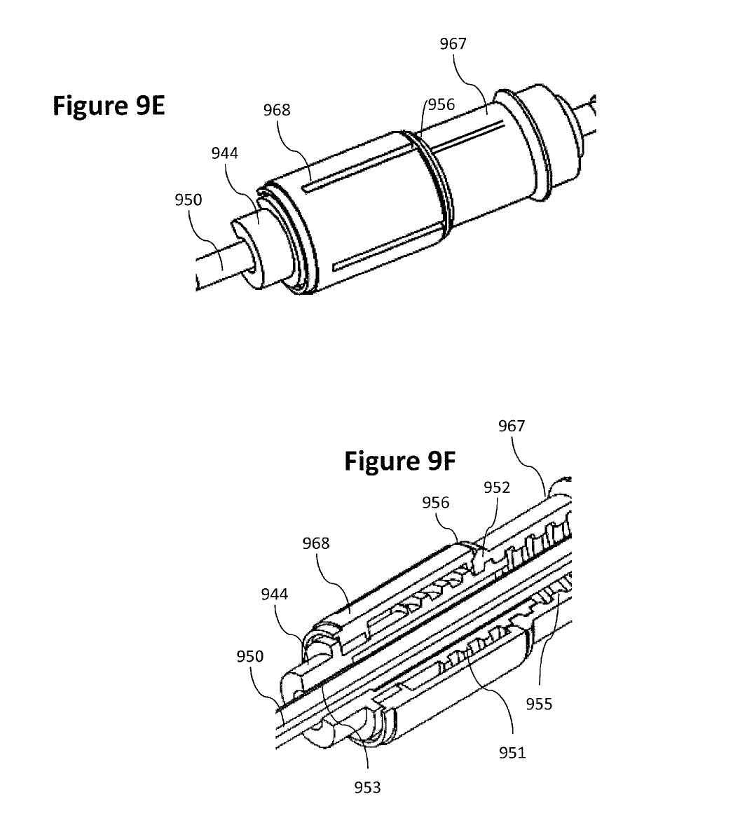

FIGS. 9A-D include detailed cross sectional side views illustrating four states of an autoinjector according to an embodiment of the present invention. In some embodiments, an injector 900 is an automated self injection device. For example the self injecting device may in some ways be similar to a pen injector. Optionally injector 900 may be loaded with a standard type syringe 946 and/or hypodermic needle 960. For example, needle 960 may be rigidly connected and/or project from a distal end of syringe 946. Needle 960 may be coaxial with syringe 946. Alternatively or additionally the axis of needle 960 may be parallel to the primary longitudinal axis 977 of syringe 946 but offset therefrom. Syringe may be loaded into injector 900 with needle 960 in a sterile state and/or covered by a sterile cover.

In some embodiments, an injector may include for example an adhesive 978 base 942. For example, adhesive 978 base 942 may assist a user to hold injector 900 steady on the skin of a patient for an extended period. For example, injector 900 may be used to give injections of volume ranging between 0.5 and 3.0 ml over a time period ranging between 30 sec to 180 sec.

Injector 900 includes for example an annular snap resistance element 952 paired to an annular driver support 956. When a linear stress increases past a threshold, the annular snap gives way and a needle 960 may optionally be retracted to a protected location.

FIG. 9A is a schematic cross sectional side view illustrating injector 900 in an enabled state (ready for activation). For example, in the enabled state an optional safety cover and/or a sterile cover may and/or an adhesive protector may have been removed from the injector. In the enabled state needle 960 is in a protected location, created by a shield 941 which extends the distal end of a housing 940 of the injector. Needle 960 and/or shield 941 may optionally be retained in position, for example by a snap and/or held in position by a biasing device, for example a spring.

In some embodiments, needle 960 may optionally be supported by a syringe 946; which is in turn supported for example by a cylindrical outer sleeve 968. Outer sleeve 968 may optionally be supported by an annular support 956 resting on an annular snap resistance element 952. For example annular snap resistance element 952 may extend radially outward from a cylindrical inner sleeve 967. Optionally, inner sleeve 967 and/or outer sleeve 968 and/or a driver 944 may be operationally linked to a transmission 984 such that rotating transmission 984 rotates one or more of inner sleeve 967 and/or outer sleeve 968 and/or a driver 944.

In some embodiments, a motor switch 982 may be located in shield 941. In the enabled state (before activation), switch 982 is optionally switched off.

In injector 900, syringe 946 is held to outer housing 940 by a socket 961. Socket 961 allows syringe 946 to slide axially with respect to housing 940 but not to move laterally. In injector 900, transmission 984 is held rotatably fixed to housing 940 by bearing 959 in a hub 965.

FIG. 9B is a schematic cross sectional side view illustrating injector 900 immediately after activation. For example, to activate the injector, a user may place the distal end of the injector (including for example an adhesive 978 and/or an activation zone on base 942) against the skin 966 of a patient and/or push 964 on the proximal end of the injector until shield 941 collapses into housing 940 in a direction parallel to the longitudinal axis of needle 960. Collapse of shield 941 may optionally unshield needle 960 tip which may for example be pushed into the skin 966 of the patient. For example, in operation, needle 960 may protrude from injector 900 into a patient. Optionally, in operation, needle 960 may be in fluid communication with syringe 946 and/or the patient. For example needle 960 may supply a fluid pathway for discharging medicine directly from syringe 946 through needle 960 into the patient.

In some embodiments, collapse of shield 941 may activate switch 982. For example in injector 900 switch 982 is depressed by being pushed against syringe 946. Depressing switch 982 may activate a motor 976 to start discharging a drug. For example, in injector 900 motor 976 turns a transmission 984. Transmission 984 may include for example a gear. Transmission 984 may optionally rotate inner sleeve 967 and/or driver 944. In exemplary injector 900, driver 944 includes teeth and/or threads which engage a screw thread 953 on a plunger rod 950. Rotating driver 944 may optionally drive plunger rod 950 and/or plunger 948 in the distal direction, discharging the medicine. Optionally, plunger 948 continues to move distally until it is stopped by for example a blockage in the fluid path (preventing further discharge) and/or until plunger 948 reaches the distal end of syringe 946. Optionally, when needle 960 is in the extended position, a flange 947 of syringe 946 seats against a bracket 973, which holds syringe 946 and/or prevents further longitudinal movement.

FIG. 9C is a schematic cross sectional side view illustrating injector 900 at the end of discharge of the payload. For example plunger 948 has discharged all of the medicine out of syringe 946 and/or has reached the distal end of syringe 946. Optionally, further rotation of driver 944 increases the stress pushing driver 944 proximally. Interference element 952 may serve as stress sensor. For example, motor 976 may supply enough torque to create a force which overcomes interference element 952.

In some embodiments, once interference element 952 is overcome a course inner threading 955 in sleeve 967 rotates with respect to a course outer thread 951 of driver 944 drawing driver 944 and/or plunger 948 and/or syringe 946 and/or needle 960 proximally into a retracted state. For example, the course thread 951 has an opposite threading from the threading 953 between driver 944 and plunger rod 950. The same direction of rotation that drives plunger 948 distally before overcoming interference element 952 also draws back plunger 948 and/or syringe 946 and/or needle 960 proximally after overcoming interference element 952. Optionally needle 960 is retracted into a protected location inside housing 940 for example as illustrated in FIG. 9D. Alternatively or additionally, course thread 951 may have an the same direction of threading as threading 953 between driver 944 and plunger rod 950 and optionally rotation may be reversed to retract needle 960.

FIG. 9D is a schematic cross sectional side view illustrating injector 900 in a safe state after finishing injection. Needle 960 point has optionally been retracted into a protected location within housing 940. Syringe 946 has optionally been retracted. In exemplary injector 900, when syringe 946 is retracted, it no longer depresses switch 982. Switch 982 may be biased off and/or raising syringe 946 may shut off motor 976.

In some embodiments one or more windows may be supplied. A user may be able to determine a status of the device by viewing for the windows. For example in FIG. 9D, injector 900 has been supplied with two windows 990a,b. For example window 990a is located such that during injection, the user views inner sleeve 967 through window 990a. When outer sleeve 968 has been retracted, it may optionally slide over inner sleeve 967. After outer sleeve 968 has been retracted, the user views outer sleeve 968 through window 990a. Optionally window 990a may serve as an indicator whether it is safe to remove the injector. For example, outer sleeve 968 may be colored green and/or driver 944 and/or inner sleeve 967 may be colored red. For example, as long as the user sees red in window 990a needle 960 tip has not been retracted and/or it is unsafe to remove the injector from the patient's skin; and/or when the user views green through window 990a needle 960 has been retracted and/or discharge has ceased and/or it is safe to remove the injector from the skin of the patient. Optionally, window 990b may be used to indicate whether an entire payload of medicine has been administered. For example, syringe 946 may be made of a transparent material. For example, during injection, the user can see the medicine through window 990b; after syringe 946 is retracted if the payload has been fully discharged then the user will view plunger 948 through window 990b. Optionally, if the user sees plunger 948 through both window 990b and outer sleeve 968 through window 990a then the user can ascertain that it is safe to remove the injector and/or that the drug was fully discharged.

In injector 900, for example, after retraction of the needle the device may be twisted such that one side of the adhesive is lifted and/or peeled (as illustrated by arrow 983 in FIG. 9D) from the skin while the far edge of the base of the injector remains in contact with the skin and serves as a fulcrum.

FIGS. 9E and 6F illustrate an external view and cut away view respectively of a needle retractor according to exemplary injector 900 of the current invention. Injector 900 may optionally be designed such that, under sufficient linear stress, support 956 of external sleeve 968 deforms and/or opens to pass over resistance element 952. Optionally, transmission 984 and/or inner sleeve 967 may be formed of one or more pieces of molded plastic. Optionally outer sleeve 968 and/or driver 944 may be formed of one or more pieces of molded plastic.

FIG. 9F, illustrates details of a rotary needle retractor according to an embodiment of the current invention. FIG. 9F illustrates driver 944 before needle retraction (for example in a secured state, an enabled state and/or an active state). In the exemplary embodiment, driver 944 is engaged by a set of fine screw threads 953 to rod 950. In the exemplary embodiment, driver 944 is engaged by a set of course screw threads 951, 955 to inner sleeve 967. Optionally, course screw threads 951, 955 are threaded in an opposite sense from fine screw threads 953.

In the exemplary embodiment, prior to needle retraction, sleeve 967, 968 and driver 944 are prevented from sliding longitudinally with respect one another. While sleeves 967, 968 and driver 944 are prevented from relative longitudinal movement, threads 951 and 955 prevent inner sleeve 967 and driver 944 from rotating with respect to one another.

In some embodiments, motor 976 drives transmission 984 to rotate inner sleeve 967. Optionally, before needle retraction, rotating inner sleeve 967 rotates driver 944. The sense of screw threads 953 and the rotating direction of motor 976 are optionally chosen such that rotating driver 944 relative to rod 950 pushes rod 950 and/or plunger 948 distally, optionally discharging a drug.

When plunger 948 has reached the distal end of syringe 946, rod 950 is prevent from further distal movement. Torque applied to driver 944 produces a strong proximal stress on driver 944 and/or outer sleeve 968. The strong proximal stress overcomes and/or releases interference element 952. Once interference element 952, is released outer sleeve 968 and/or driver 944 can move longitudinally with respect to inner sleeve 967. Further rotation of inner sleeve 967 rotates sleeve 967 with respect to driver 944. The sense of screw threads 955 and 951 and the rotating direction of motor 976 are optionally chosen such rotating driver 944 relative to sleeve 967 draws driver 944 and/or rod 950 and/or plunger 948 and/or syringe 946 and/or needle 960 proximally, optionally retracting needle 960. Optionally the pitch of screw threads 951, 953 and/or 955 can be tuned to achieve a desired rate of medicine discharge and/or needle retraction for a given rotation rate of the motor. In some embodiments, as rod 950 and/or plunger 948 are drawn proximally, friction between plunger 948 and syringe 946 draws syringe 946 and/or needle 960 proximally. Alternatively or additionally, outer sleeve 968 may be attached to syringe 946. Drawing hack on driver 944 may draw outer sleeve 968 and syringe 946 back with it. In some embodiments additional threaded elements may be added to produce a multi-part telescoping assembly for extending plunger 948 to discharge medicine and/or for retracting needle 960. In some embodiments some or all of rod 950, inner sleeve 967, and/or outer sleeve 968 and/or transmission 984 may be formed of molded plastic and or other materials.

### 10 Stabilized Pen Injector

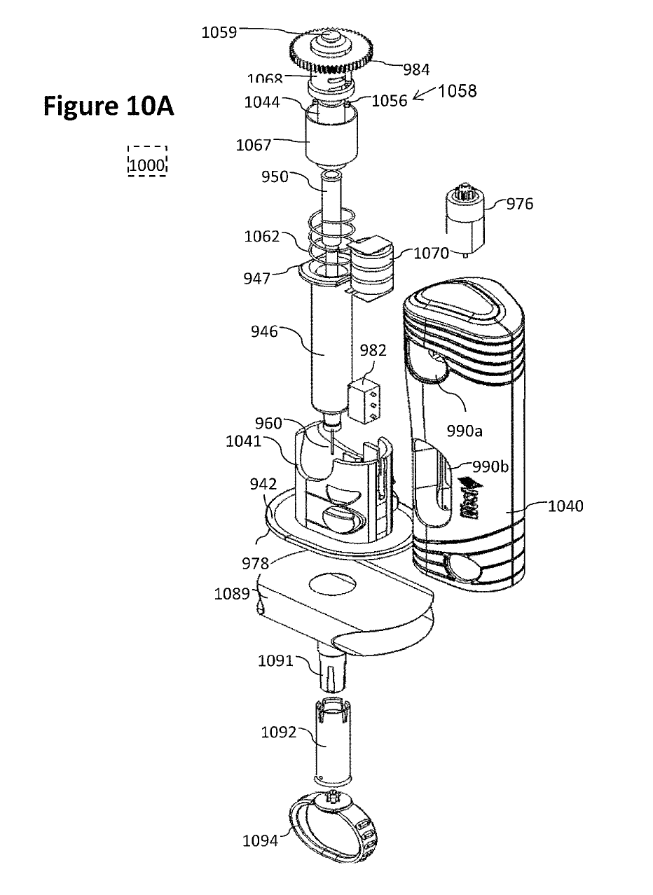

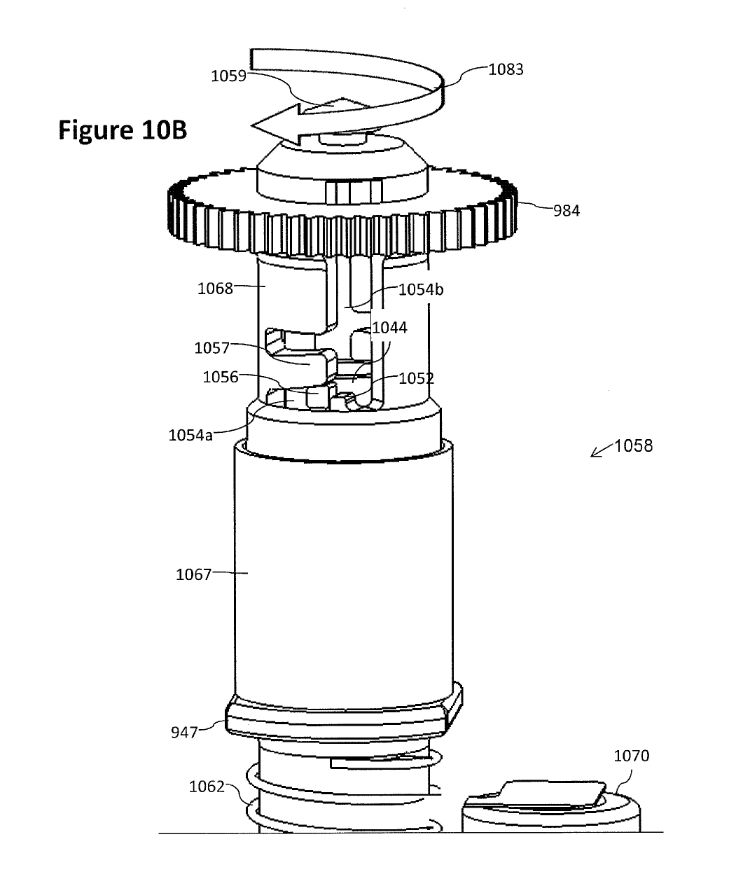

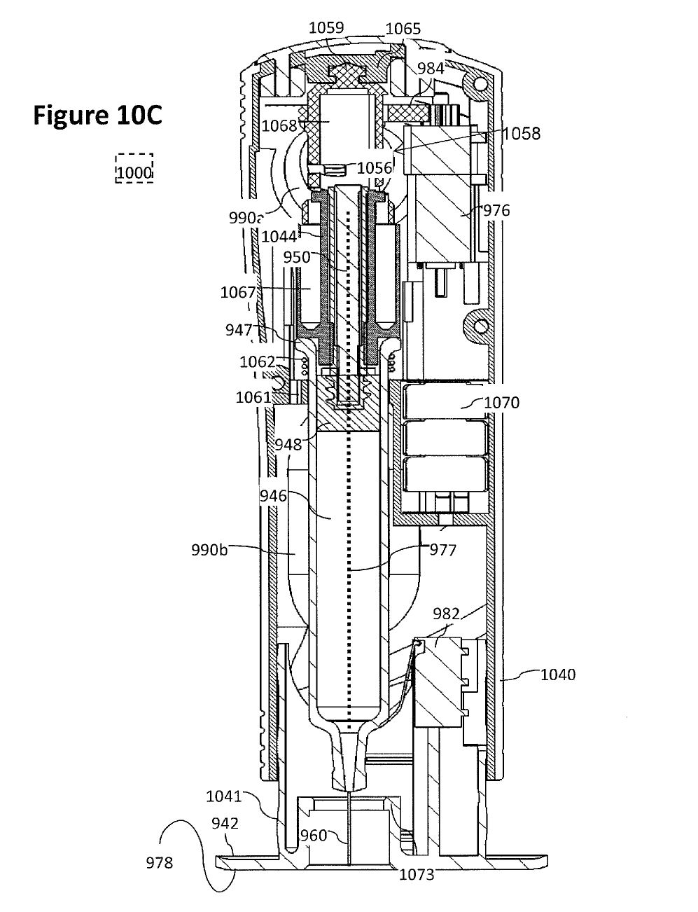

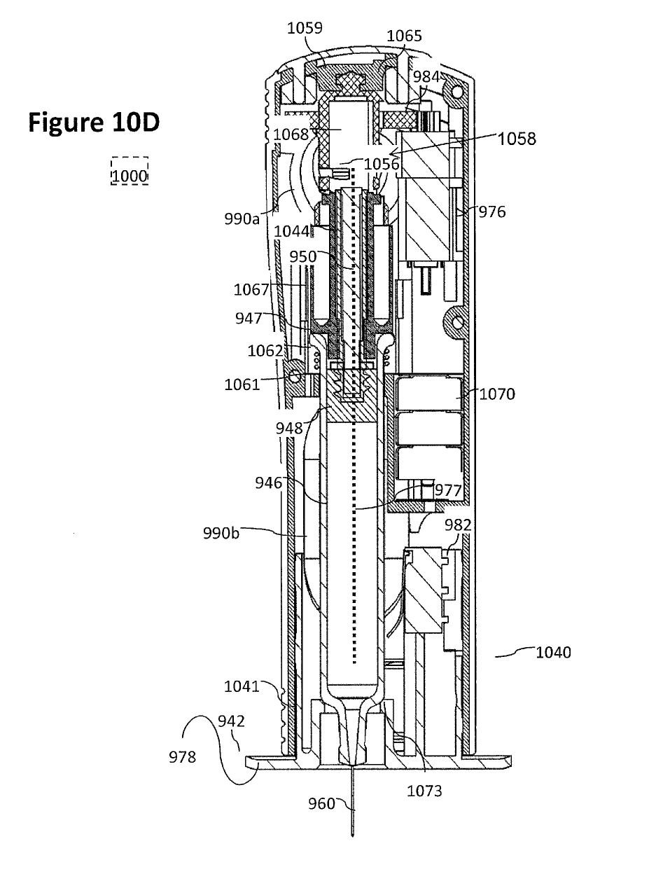

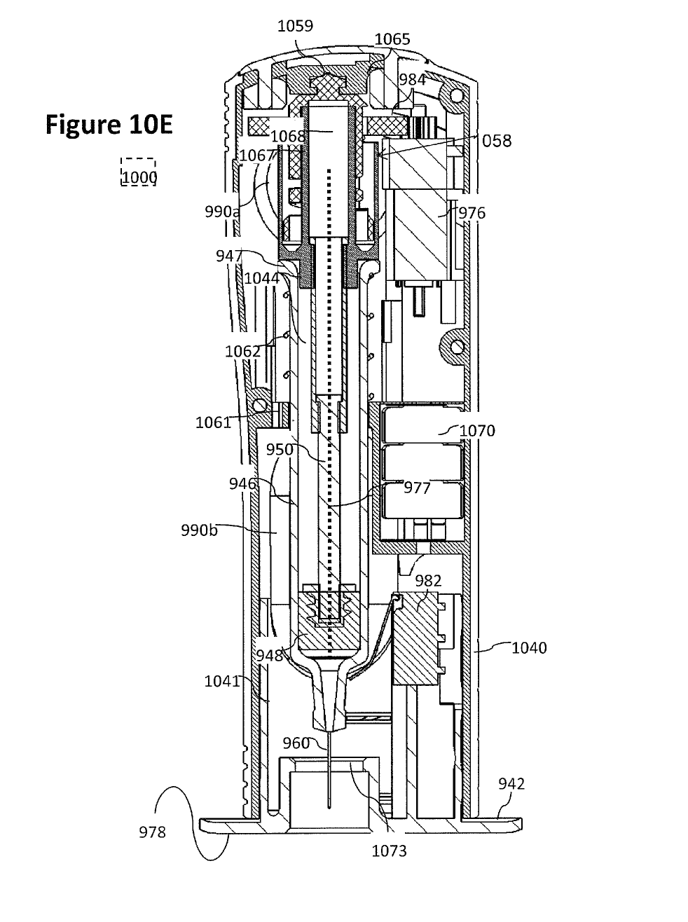

FIGS. 10A-H illustrate a stabilized injector 1000 according to some embodiments of the present invention. Exemplary injector 1000 is an automated injection device in some ways similar to a pen injector. Optionally injector 1000 may be loaded with a standard type syringe 946 and/or hypodermic needle 960. Optionally syringe 946 may be supplied loaded with medicine and/or covered with a sterile needle cover 1091. Syringe 946 may be loaded into injector 1000 with in a sterile state with needle cover 1091 in place. Injector 1000 may include for example an adhesive 978 base 942. In some embodiments, adhesive 978 base 942 may assist a user to hold injector 1000 steady on the skin of a patient for an extended period. For example, injector 1000 may be used to give injections of volume ranging between 0.5 and 3.0 ml over a time period ranging between 30 sec to 180 sec.

FIG. 10A illustrates an exploded view of injector 1000. Some components of the exemplary embodiment of injector 1000 which are similar to corresponding parts of the exemplary embodiment of injector 900 are marked with the same number as the corresponding parts of the exemplary embodiment of injector 900.

In the exemplary embodiment of injector 1000 a power supply (for example batteries 1070) may optionally supply power to gear motor 976. FIGS. 10A,B illustrate flange 947 of syringe 946. Optionally flange 947 has at least one non-rounded edge which may be held inside an autoinjector (for example autoinjectors 400, 900 and/or 1000) preventing rotation of syringe 946. Outer housing 1040 and/or shield 1041 of injector 1000 are similar to outer housing 940 and/or shield 941 of injector 900.

Some embodiments of a stabilized autoinjector (for example as illustrated in injector 1000 but optionally included in injectors 900 and/or embodiments 400 and/or 200 and/or 100) may include a safety cover and/or an adhesive protector and/or a handle.

FIG. 10B illustrates exemplary retraction mechanism 1058. Retraction mechanism 1058 is optionally activated by a combination of torque and linear stress. For example retraction mechanism 1058 is optionally activated may when plunger 948 is blocked for example when it reaches the end of injection (for example as described in regards to FIGS. 9A-D and/or due to an occlusion of needle 960).

In some embodiments, during drug discharge a motor (for example motor 976) rotates transmission 984 in the direction of arrow 1083. Transmission 984 may optionally be rigidly connected to and/or integrally molded with inner sleeve 1068. Rotating transmission 984 may also rotate inner sleeve 1068. A pin 1056 protrudes from driver 1044 into a nearly lateral slot 1054a in sleeve 1068. While pin 1056 is in slot 1054a, driver 1044 is prevented from moving longitudinally with respect to inner sleeve 1068. In some embodiments syringe 946 is supported (from moving proximally) by driver 1044.

In some embodiments, when there is a strong linear force on driver 1044 in the proximal direction and/or there is a strong torque on sleeve 1068 in the direction of arrow 1083, arm 1057 is deflected upward and pin 1056 slides past an interference element 1052 into a longitudinal slot 1054b. In slot 1054b pin 1056 may slide longitudinally (in the proximal direction). A geometry of pin 1056 and/or interference element 1052 may be chosen to achieve a desired resistance to movement. For example, pin 1056 and/or interference element 1052 may have a squared side, a flat side, a rounded side etc.

In some embodiments, a spring (for example spring 1062) biases syringe 946 in the proximal direction. For example spring 1062 may apply a proximal force to flange 947. Optionally another biasing element may be used in place of spring 1062. For example, a biasing element may include a stretched element (for example a rubber band and/or a twisted elements and/or a deflected plastic element).

Optionally when pin 1056 enters longitudinal slot 1054b, spring 1062 pushes syringe 946 and/or outer sleeve 1067 and/or needle 960 and/or driver 1044 and/or pin 1056 proximally, retracting needle 960. Optionally, needle 960 may be held in the retracted position by spring 1062. Alternatively or additionally a locking mechanism may be included to lock needle 960 in the retracted position, for example, a one way catch and/or an interference element may lock against syringe 946 as it is retracted and/or against pin 1056 in slot 1054b. Optionally, in injector 1000 driver 1044 includes two molded plastic telescoping pieces. One piece is optionally integrally molded with outer sleeve 1067. Optionally, sleeve 1067 and/or driver 1044 may be made as a single piece and/or multiple parts. They may be formed of plastic and/or another material and/or they may be molded and/or formed by another process.

FIG. 10C illustrates exemplary embodiment 1000 assembled and/or in an enabled state before insertion of needle 960 into a patient. FIG. 10C illustrates various optional details and/or supporting structures for syringe 946 and/or plunger.

In injector 1000, syringe 946 is held to outer housing 1040 by a socket 1061. Socket 1061 allows syringe 946 to slide axially with respect to housing 1040 but not to move laterally. In injector 1000, transmission 984 is held rotatably fixed to housing 1040 by bearing 1059 in a hub 1065.

FIG. 10D illustrates exemplary injector 1000 in an active state. For example when the injector is in the enabled state, a user may place adhesive against the skin of a patient and push downward (distally) on housing 1040. Housing 1040 and its contents (for example syringe 946, transmission 984, locking assembly of retraction mechanism 1058 etc.) along with needle 960 are all pushed distally along the axis of needle 960. As needle 960 moves distally, the needle tip passes through a hole in shield 1041. For example, in operation, needle 960 may protrude from injector 900 into a patient. Optionally, in operation, needle 960 may be in fluid communication with syringe 946 and/or the patient. For example needle 960 may supply a fluid pathway for discharging medicine directly from syringe 946 through needle 960 into the patient.

In some embodiments, when needle 960 is in the extended position, the front end of syringe 946 seats into a bracket 1073. Bracket 1073 may optionally hold syringe 946 steady and/or prevents further longitudinal movement and/or prevent lateral movement with respect to housing 1040.

FIG. 10E illustrates injector 1000 after needle retraction. Optionally driver 944 includes a telescopic assembly, which is shown in an extended state in FIG. 10E. Optionally, after retraction of needle 960, the entire device may be twisted to peel adhesive 978 from the skin.

Various aspects or features illustrated herein with respect to a particular embodiment may be combined with other embodiments. For example, needle 460 and 560 of embodiment 400 and 500 are shown mounted at an angle to base and/or activation zone 442 or 542. Alternatively or additionally they may be perpendicular to the base. For example, needles 960 of embodiments 900 and 1000 are shown perpendicular to base 942. Alternatively or additionally they may be at an angle to the base. Needle covers and/or protective covers illustrated in one embodiment may be used with another embodiment. Retraction mechanisms illustrated in one embodiment may be used with another embodiment. A clip, an interference element, a catch and/or another locking mechanism may hold an injector in one or another state. For example an interference element may hold a needle in a retracted position and/or in the extended position.

### 11 Manual Needle Safeguard

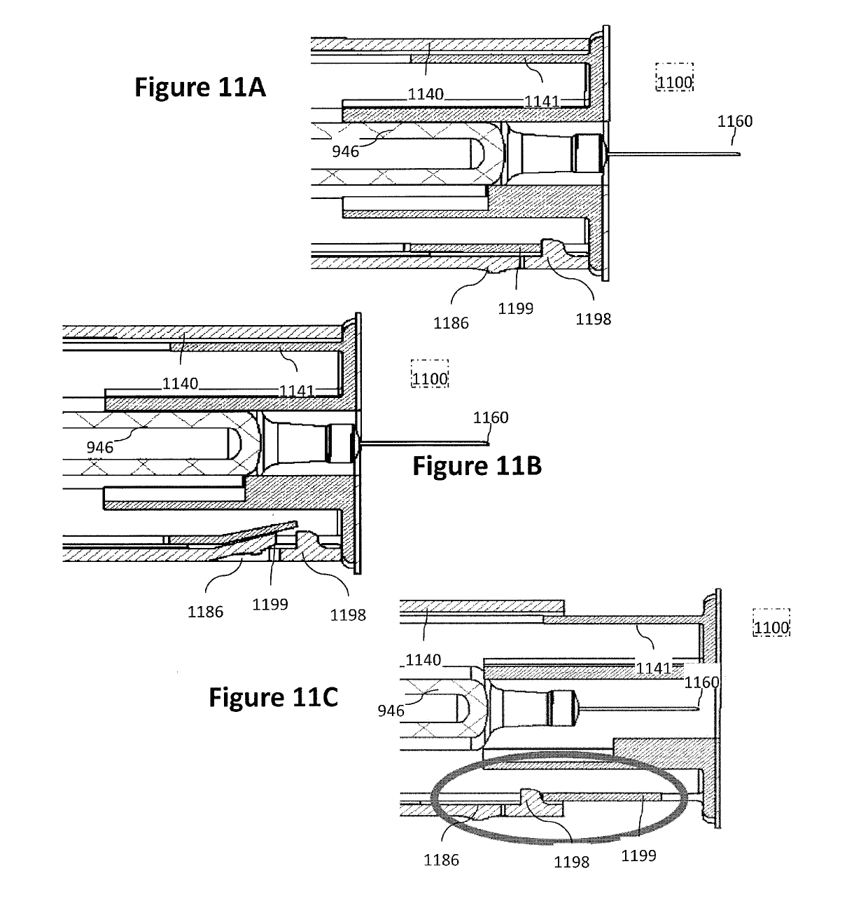

FIGS. 11A-C illustrate a manual trigger for a needle protector of an autoinjector according to an embodiment 1100 of the present invention. In some embodiments a user may want to abort administration of a drug by an autoinjector and/or safeguard a needle of the autoinjector (for example when the automatic safeguard mechanism fails). Embodiment 1100 supplies a manual system for triggering a needle shield at a user's discretion.

FIG. 11A illustrates an autoinjector in an activated state. In the active state, a needle tip 1160 may be unshielded. For example, in FIG. 11A needle tip 1160 is shown extending out the proximal end of the injector. Optionally, a needle shield 1141 may be pushed distally into a housing 1140 of the injector unshielding needle tip 1160. For example, in embodiment 1100 shield 1141 is held back in housing 1140 by a catch 1199 abutting against the distal side of a latch 1198.

FIG. 11B illustrates initiation of manual release of a needle shield according to an embodiment 1100. For example, a user presses inward on a needle guard release 1186. Optionally, pushing needle guard release 1186 disengages catch 1199 from latch 1198. For example, needle shield 1141 may be biased to deploy outward (proximally) when catch 1199 is disengaged. The pressing needle guard release 1186 extends the needle guard and/or housing around the needle, retracting the needle point into a shielded location.

FIG. 11C illustrates an exemplary result of manual extension of the housing and/or retraction of the needle in exemplary embodiment 1100 of the present invention. Needle shield 1141 is shown in an exemplary deployed state. For example catch 1199 may abut on the proximal side of latch 1186 locking shield 1141 in the deployed state. Optionally, needle tip 1160 is safeguarded in a protected space surrounded by deployed needle shield 1141.

### 12 Rotation Resistance

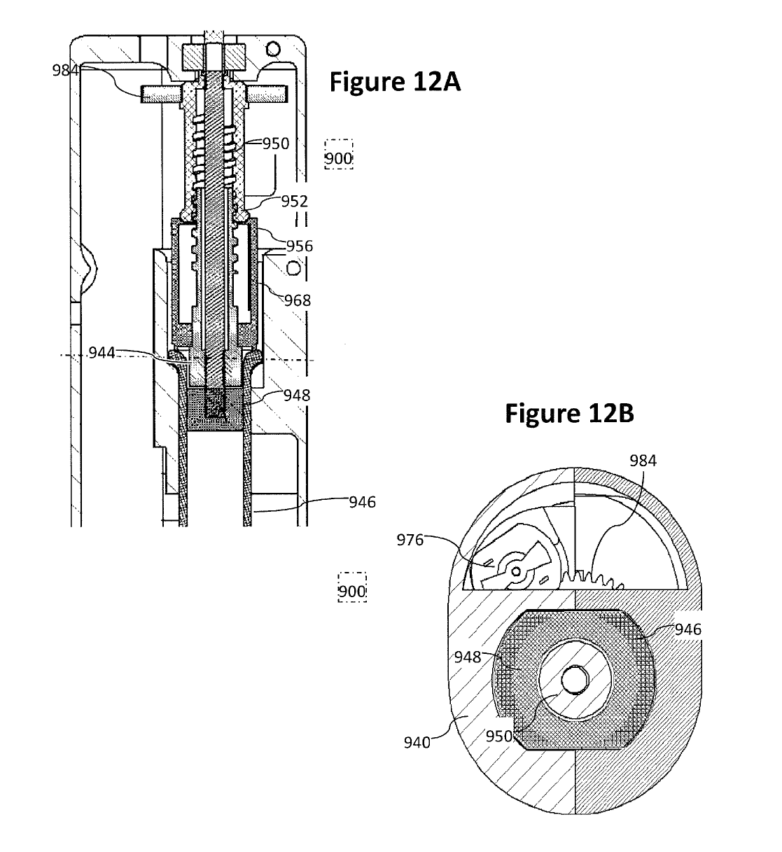

FIGS. 12A,B illustrate an optional non-circular cross section for syringe 946 according to an embodiment of the present invention. Optionally syringe 946 may be prevented from rotating for example by giving it a non-circular cross section.

FIG. 12A is a side view of embodiment 900 showing a cutting plane for the cross sectional view of FIG. 12B.

FIG. 12B is a cross section view of embodiment 900 illustrating and optional non-circular cross section of syringe 946.

### 13 Microswitch Activator for an Autoinjector

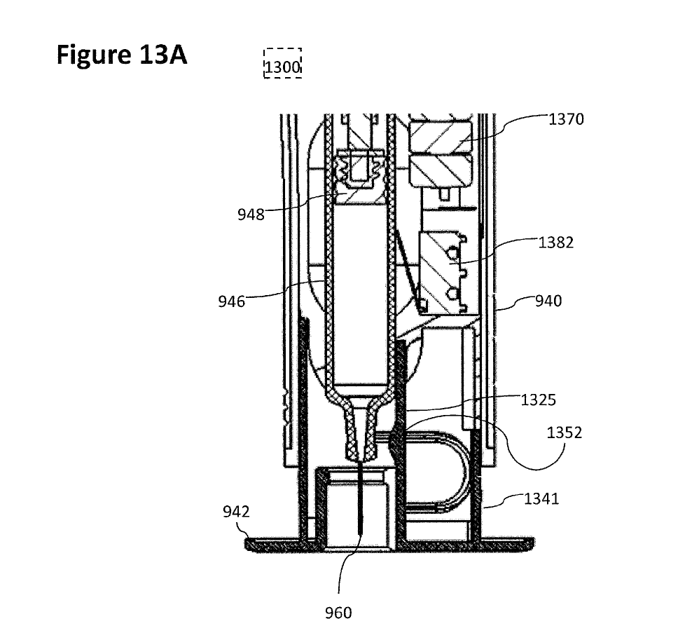

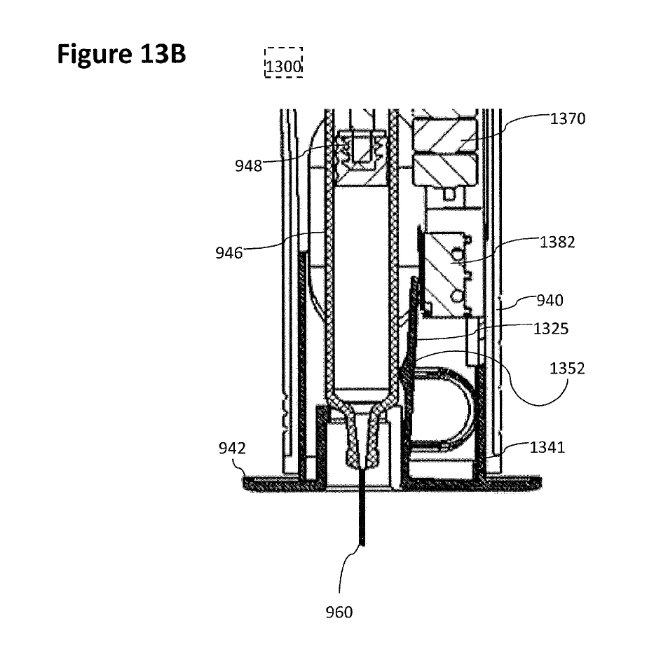

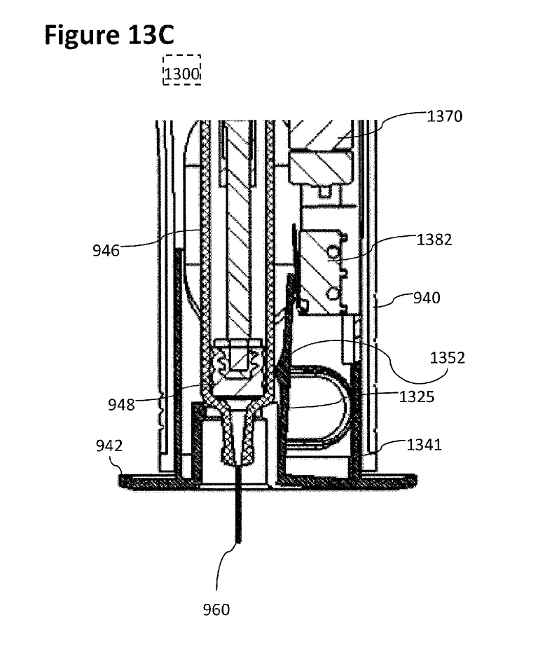

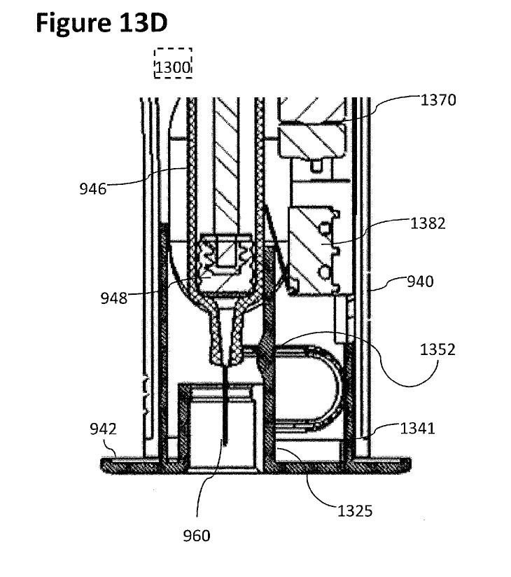

FIGS. 13A-D is are cutaway illustrations of an exemplary embodiment of an autoinjector 1300 with a motor switch located in the main body of the injector in accordance with some embodiments of the current invention. Optionally the motor switch remains stationary with respect to the housing of the autoinjector, for example housing 940, and/or with respect to and/or a motor and/or a power source, for example batteries 1370. When an activator of the injector is triggered (for example by collapsing a shield 1341), movement of the activator may activate the switch. When the needle 960 is retracted and/or shielded (for example after injection), the switch is optionally returned to the inactivated state. Keeping a switch stationary with respect to a motor and/or power source may simplify assembly of the auto injector.

FIG. 13A illustrates injector 1300, prior to activation in accordance with an embodiment of the current invention. Optionally many structures are the same as other embodiments listed herein above or below, for example, injector 1300 may include the advancement and/or retraction mechanism of injector 900. Optionally, prior to activation, switch 1382 is in a deactivated position.

FIG. 13B illustrates injector 1300, immediately after activation in accordance with an embodiment of the current invention. In some embodiments, an injector may be activated by collapsing a needle shield 1341. Collapsing shield 1341 optionally exposes needle 960 and/or pushes an element 1352 against a side of a syringe and/or pushes an extension 1325 of shield 1341 into switch 1382 and/or activates switch 1382 and/or activates a motor (for example similar to motor 976 of injector 900).

FIG. 13C illustrates injector 1300, immediately at the end of injection in accordance with an embodiment of the current invention. Optionally for as long as shield 1341 remains in a collapsed state and/or needle 960 remains in an extended state (protruding from the injector), extension 1325 continues to hold switch 1382 in an activated state and/or a motor continues to drive plunger 948. Optionally, when plunger 948 reaches the end of syringe 946 a retraction mechanism (for example lock 758) retracts syringe 946 and/or needle 960 to a retracted state (as illustrated for example in FIG. 13D).

FIG. 13D illustrates injector 1300 in a retracted state in accordance with an embodiment of the current invention. Optionally, when needle 960 and/or syringe 946 is retracted, element 1352 is released and/or extension 1325 moves away from switch 1382 and/or switch 1382 moves into a deactivated state and/or the motor is switched off.

It is expected that during the life of a patent maturing from this application many relevant technologies will be developed and the scope of the terms are intended to include all such new technologies a priori.

As used herein the term “about” refers to ±5%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A drug injector having a needle safeguard mechanism comprising:
a housing including a shielded location;
a point of the needle protruding from said housing;
a retractor mechanically coupled to the needle, said retractor retracting said point to said shielded location; and
a sensor operationally linked to said retractor, said sensor sensing a resistance to discharging of the drug through the needle;
wherein said retractor is responsive to said sensor to retract said point to said shielded location in response to said resistance.

2. The drug injector of claim 1, wherein said retractor includes a support of the needle and wherein said support reverts to a retracted state in response to said resistance.

3. The drug injector of claim 2, wherein said support includes an interference element, and wherein said resistance overcomes said interference element.

4. The drug injector of claim 3, wherein said interference element includes an annular snap.

5. The drug injector of claim 2, wherein said support includes a telescoping assembly.

6. The drug injector of claim 5, wherein said telescoping assembly is held in an extended mode by an interference element.

7. The drug injector of claim 2, further comprising:
a driver driving said discharging and wherein said driver is supported by said support, and wherein in response to said resistance said driver produces a stress on said support and wherein said stress reverts said support to said retracted state.

8. The drug injector of claim 7, further including:
a plunger and wherein said driver drives said discharging by imparting a linear motion to said plunger and wherein said resistance is to said linear motion of said plunger.

9. The drug injector of claim 1 further comprising:
a lock mechanically coupled to the needle, said lock retaining said point in said protruding position; and wherein said retractor is operationally linked to said lock to release said lock in response to said resistance.

10. The drug injector of claim 9, wherein said sensor is further sensitive to a second condition, and said lock retains said point in said protruding position until said second condition is met.

11. The drug injector of claim 10, wherein said point is biased to said shielded position.

12. The drug injector of claim 9, wherein said lock includes at least one element selected from the group consisting of an interference element, an annular snap, a rib snap, a flange and a groove.

13. The drug injector of claim 10, wherein said second condition is a volume of said discharging passing a predetermined threshold.

14. The drug injector of claim 1, wherein the sensor senses a torque.

15. The drug injector of claim 1, wherein said retracting is in response to a change in said resistance.

16. The drug injector of claim 1, wherein said retracting is in response to an increase in said resistance.

17. The drug injector of claim 1, wherein said retracting is in response to a decrease in said resistance.

18. The drug injector of claim 1, wherein said retracting is in response said resistance passing a threshold.

19. A method of safeguarding a needle of an injector comprising:
discharging a drug through the needle;
sensing a resistance to said discharging, and
safeguarding the needle in response to said resistance.

20. The method of claim 19, wherein said safeguarding is in response to said resistance passing a predetermined threshold.

21. The method of claim 19 wherein said safeguarding is in response to a change in said resistance.

22. The method of claim 19 wherein said safeguarding is in response to an increase in said resistance.

23. The method of claim 19 wherein said safeguarding is in response to a decrease in said resistance.

24. The method of claim 19, further comprising:
locking the needle in an extended position during said discharging; and
wherein said safeguarding includes releasing said locking.

25. The method of claim 19, wherein said resistance occurs upon at least one event selected from the group consisting of a completion of said discharging, a fluid leak, jamming of a mechanical component, disconnection of a mechanical component and obstruction of a fluid flowpath.

26. The method of claim 19, further comprising:
inhibiting said safeguarding until a second condition is met.

27. The method of claim 26, wherein said second condition includes said discharging passing a predetermined volume.

28. The method of claim 27, wherein said volume of discharging is measured by counting motor rotations.

29. The method claim 19, wherein the needle is a component of a pre filled syringe inside autoinjector and further comprising:
placing the autoinjector on a patient;
extending the needle to an extended position subsequent to said placing and prior to said discharging, and
wherein said safeguarding includes retracting the needle to a retracted position.

30. The method claim 19, further comprising:
indicating that it is safe to remove an injection device including said needle after said safeguarding.
